(19) United States
(12) Patent Application Publication
(10) Pub. No.: US 2020/0046322 A1
(43) Pub. Date: Feb. 13, 2020

(54) METHODS AND APPARATUSES FOR DETERMINING AND DISPLAYING LOCATIONS ON IMAGES OF BODY PORTIONS BASED ON ULTRASOUND DATA

(71) Applicant: Nathan Silberman, Brooklyn, NY (US)

(72) Inventor: Nathan Silberman, Brooklyn, NY (US)

(73) Assignee: Butterfly Network, Inc., Guilford, CT (US)

(21) Appl. No.: 16/533,090

(22) Filed: Aug. 6, 2019

Related U.S. Application Data
(60) Provisional application No. 62/715,778, filed on Aug. 7, 2018.

(51) Int. Cl. A61B 8/00; A61B 8/08

(52) U.S. Cl. CPC A61B 8/469; A61B 8/5246

(57) ABSTRACT

Aspects of the technology described herein relate to displaying locations on images of body portions. Based on ultrasound data collected from a subject by an ultrasound device, a first location on an image of a body portion may be determined. The first location on the image of the body portion may correspond to a current location of the ultrasound device relative to a subject where the ultrasound device collected the ultrasound data. A first marker may be displayed on the image of the body portion at the first location. A second location on the image of the body portion may be determined, where the second location on the image of the body portion corresponds to a target location of the ultrasound device relative to the body portion of the subject. A second marker on the image of the body portion at the second location may be displayed.

### CROSS-REFERENCE TO RELATED APPLICATIONS

This application claims the benefit under 35 U.S.C. § 119(e) of U.S. Provisional Patent Application Ser. No. 62/715,778, filed Aug. 7, 2018 under Attorney Docket No. B1348.70086US00 and entitled “METHODS AND APPARATUSES FOR DETERMINING AND DISPLAYING LOCATIONS ON IMAGES OF BODY PORTIONS BASED ON ULTRASOUND DATA,” which is hereby incorporated herein by reference in its entirety.

### FIELD

Generally, the aspects of the technology described herein relate to determining and displaying locations on images of body portions based on ultrasound data.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using an ultrasound device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus includes a processing device in operative communication with an ultrasound device, the processing device configured to determine, based on first ultrasound data collected from a body portion of a subject by the ultrasound device, a first location on an image of a body portion, wherein the first location on the image of the body portion corresponds to a current location of the ultrasound device relative to the body portion of the subject where the ultrasound device collected the first ultrasound data; and display a first marker on the image of the body portion at the first location.

In some embodiments, the processing device is configured, when displaying the first marker on the image of the body portion, to display the first marker on a display screen of the processing device. In some embodiments, the processing device is further configured to receive the first ultrasound data from the ultrasound device. In some embodiments, the processing device is further configured to update the first location of the first marker as further ultrasound data is received at the processing device from the ultrasound device. In some embodiments, the processing device is further configured to determine a second location on the image of the body portion, wherein the second location relative to the image of the body portion corresponds to a target location of the ultrasound device relative to the body portion of the subject; and display a second marker on the image of the body portion at the second location. In some embodiments, the processing device is configured, when determining the second location, to receive a selection of the second location on the image of the body portion. In some embodiments, the processing device is configured, when displaying the second marker, to display the second location on a display screen of the processing device. In some embodiments, the processing device is further configured to receive a selection of an anatomical view associated with the target location. In some embodiments, the processing device is further configured to provide an instruction for moving the ultrasound device from the current location to the target location. In some embodiments, the processing device is further configured to provide an indication that the current location is substantially equal to the target location. In some embodiments, the processing device is further configured to determine, based on second ultrasound data collected from the body portion of the subject by the ultrasound device at a past time, a second location on the image of the body portion, wherein the second location on the image of the body portion corresponds to a past location of the ultrasound device relative to the body portion of the subject where the ultrasound device collected the second ultrasound data; and display a path on the image of the body portion that includes the first location and the second location. In some embodiments, the body portion comprises a torso.

According to another aspect, an apparatus includes processing circuitry configured to receive a selection of a location on an image of a body portion and automatically retrieve ultrasound data that was collected by an ultrasound device at a location relative to a subject corresponding to the selected location.

In some embodiments, the processing circuitry is further configured to display, on the image of the body portion, one or more markers at a plurality of locations on the image of the body portion. In some embodiments, the processing circuitry is further configured to determine the plurality of locations on the image of the body portion, wherein each respective location of the plurality of locations corresponds to a location relative to the body portion of a subject where an ultrasound device collected a respective set of ultrasound data of a plurality of sets of ultrasound data. In some embodiments, the processing circuitry is further configured to receive a selection of the plurality of sets of ultrasound data. In some embodiments, the plurality of sets of ultrasound data comprise a set of ultrasound data containing an anatomical view of a proximal abdominal aorta, a set of ultrasound data containing an anatomical view of a mid abdominal aorta, and a set of ultrasound data containing an anatomical view of a distal abdominal aorta. In some embodiments, the processing circuitry is configured, when displaying the one or more markers at the plurality of locations, to display a plurality of discrete markers at each of the plurality of locations. In some embodiments, the processing circuitry is configured, when receiving the selection of the location on the image of the body portion, to receive a selection of a marker of the plurality of discrete markers. In some embodiments, the processing circuitry is configured, when retrieving the ultrasound data corresponding to the selected location, to retrieve ultrasound data that was collected at a location relative to the subject corresponding to a location of the selected marker on the image of the body portion. In some embodiments, the processing circuitry is configured, when displaying the one or more markers at the plurality of locations, to display a path along the plurality of locations. In some embodiments, the processing circuitry is configured, when receiving the selection of the location on the image of the body portion, to receive a selection of a location along the path. In some embodiments, the processing circuitry is configured, when retrieving the ultrasound data corresponding to the selected location, to retrieve ultrasound data that was collected at a location relative to the subject corresponding to the selected location along the path. In some embodiments, the path extends along an abdominal aorta of the body portion in the image. In some embodiments, the body portion comprises a torso.

According to another aspect, an apparatus includes processing circuitry configured to receive a selection of ultrasound data, determine a location on an image of a body portion corresponding to a location relative to the body portion of a subject where an ultrasound device collected the ultrasound data, and display, on the image of the body portion, a marker at the determined location.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example coordinate system for a canonical body portion, more specifically a canonical torso, in accordance with certain embodiments described herein;

FIG. 2 illustrates an example process for guiding collection of ultrasound data, in accordance with certain embodiments described herein;

FIG. 3 illustrates an example graphical user interface (GUI) that may be displayed on a display screen of a processing device in an ultrasound system, in accordance with certain embodiments described herein;

FIG. 4 illustrates an example GUI that may be displayed on a display screen of a processing device in an ultrasound system, in accordance with certain embodiments described herein;

FIG. 5 illustrates an example GUI that may be displayed on a display screen of a processing device in an ultrasound system, in accordance with certain embodiments described herein;

FIG. 6 illustrates an example GUI that may be displayed on a display screen of a processing device in an ultrasound system, in accordance with certain embodiments described herein;

FIG. 7 illustrates an example GUI that may be displayed on a display screen of a processing device in an ultrasound system, in accordance with certain embodiments described herein;

FIG. 8 illustrates an example process for retrieving ultrasound data, in accordance with certain embodiments described herein;

FIG. 9 illustrates an example GUI that may be displayed on a display screen of a processing device in an ultrasound system, in accordance with certain embodiments described herein;

FIG. 10 illustrates an example GUI that may be displayed on a display screen of a processing device in an ultrasound system, in accordance with certain embodiments described herein;

FIG. 11 illustrates an example GUI that may be displayed on a display screen of a processing device in an ultrasound system, in accordance with certain embodiments described herein;

FIG. 12 illustrates an example GUI that may be displayed on a display screen of a processing device in an ultrasound system, in accordance with certain embodiments described herein;

FIG. 13 illustrates an example process for retrieving ultrasound data, in accordance with certain embodiments described herein;

FIG. 14 illustrates an example GUI that may be displayed on a display screen of a processing device in an ultrasound system, in accordance with certain embodiments described herein;

FIG. 15 illustrates an example GUI that may be displayed on a display screen of a processing device in an ultrasound system, in accordance with certain embodiments described herein;

FIG. 16 illustrates an example process for collection of ultrasound data, in accordance with certain embodiments described herein;

FIG. 17 illustrates an example GUI that may be displayed on a display screen of a processing device in an ultrasound system, in accordance with certain embodiments described herein;

FIG. 18 illustrates an example GUI that may be displayed on a display screen of a processing device in an ultrasound system, in accordance with certain embodiments described herein;

FIG. 19 illustrates an example GUI that may be displayed on a display screen of a processing device in an ultrasound system, in accordance with certain embodiments described herein

FIG. 20 illustrates a schematic block diagram illustrating aspects of an example ultrasound system upon which various aspects of the technology described herein may be practiced;

FIG. 21 illustrates a schematic block diagram illustrating aspects of another example ultrasound system upon which various aspects of the technology described herein may be practiced; and

FIG. 22 illustrates an example convolutional neural network that is configured to analyze an image.

### DETAILED DESCRIPTION

Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a few inches too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include, for example, capturing ultrasound images of the incorrect anatomical structure and capturing foreshortened (or truncated) ultrasound images of the correct anatomical structure.

Conventional ultrasound systems are large, complex, and expensive systems that are typically only purchased by large medical facilities with significant financial resources. Recently, cheaper and less complex ultrasound devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. 2017/0360397 A1, which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound devices makes them more accessible to the general populace, people who could make use of such devices have little to no training for how to use them. For example, a small clinic without a trained ultrasound technician on staff may purchase an ultrasound device to help diagnose patients. In this example, a nurse at the small clinic may be familiar with ultrasound technology and physiology, but may know neither which anatomical views of a patient need to be imaged in order to identify medically-relevant information about the patient nor how to obtain such anatomical views using the ultrasound device. In another example, an ultrasound device may be issued to a patient by a physician for at-home use to monitor the patient's heart. In all likelihood, the patient understands neither physiology nor how to image his or her own heart with the ultrasound device. Accordingly, the inventors have developed assistive ultrasound imaging technology for guiding an operator of an ultrasound device how to move the ultrasound device relative to a subject in order to capture medically relevant ultrasound data.

The inventors have recognized that it may be helpful to display, on an image of a body portion (where a body portion may include a whole body), a marker (or visual indicator) indicating where on or relative to a subject an ultrasound device is currently located. The location of the marker on the image of the body portion may be based on ultrasound data collected by the ultrasound device at its current location. It may also be helpful to display on the image of the body portion a marker indicating a target location on the subject for the ultrasound device, for example, a location on the subject where a target anatomical view can be collected by the ultrasound device. An instruction may be provided for moving the ultrasound device from its current location to the target location, and as the ultrasound device moves, the marker indicating its current position may move on the image accordingly. As an example, a user of the ultrasound device may position the ultrasound device on the subject, and then view a non-ultrasound image of the subject having a marker indicating the location of the ultrasound device and the target location of the ultrasound device. The user may use this visual depiction to aid in moving the ultrasound device to the target location, in response to an instruction to do so or otherwise.”

To determine the location for the marker on the image of the body portion, it may be helpful to model the body portion and identify points on the model using a coordinate system of the model. For example, a model of a torso may be a cylinder, and points on the cylinder may be identified using a cylindrical coordinate system and certain points on the cylinder may correspond to points on the canonical torso. Ultrasound data may be inputted to a deep learning model trained to determine a set of coordinates in the coordinate system of the model that corresponds to the ultrasound data. The set of coordinates corresponding to ultrasound data may be indicative of the location on the subject where the ultrasound device collected the ultrasound data. If ultrasound data is inputted to the deep learning model in real-time, then the current set of coordinates outputted by the deep learning model may be indicative of the current location of the ultrasound device on the subject. The set of coordinates may be used to determine the location for the marker on the image of the body or body portion. If a target set of coordinates corresponding to a target location is known, an instruction may be determined based on the current set of coordinates and the target set of coordinates for moving the ultrasound device from its current location to the target location. In particular, the instruction may be determined based on which movements of the ultrasound device may result in minimization of differences between the current set of coordinates and the target set of coordinates.

Additionally, after multiple sets of ultrasound data (e.g., multiple ultrasound images) have been collected, locations on the image of the body portion corresponding to each set of ultrasound data may be determined, and markers may be displayed on the image based on those locations. To do this, a set of coordinates may be determined for each set of ultrasound data, and each set of coordinates may be used to determine a location on the image for displaying a marker. A user may select a marker and the display screen may display the particular ultrasound data collected at a location indicated by the marker. A user may also select ultrasound data and the display screen may display a marker on an image of a body portion that indicates the location on a subject where an ultrasound imaging device collected the ultrasound data. To do this, a set of coordinates may be determined for the ultrasound data, and the set of coordinates may be used to determine a location on the image for displaying a marker.

As referred to herein, a body portion should be understood to mean any anatomical structure(s), anatomical region(s), or an entire body. For example, the body portion may be the abdomen, arm, breast, chest, foot, genitalia, hand, head, leg, neck, pelvis, thorax, torso, or entire body.

As referred to herein, a device displaying an item (e.g., an arrow on an augmented reality display) should be understood to mean that the device displays the item on the device's own display screen, or generates the item to be displayed on another device's display screen. To perform the latter, the device may transmit instructions to the other device for displaying the item.

As referred to herein, collecting an ultrasound image should be understood to mean collecting raw ultrasound data from which the ultrasound image can be generated. Collecting an anatomical view should be understood to mean collecting raw ultrasound data from which an ultrasound image, in which the anatomical view is visible, can be generated.

In some embodiments described herein, a location on an image of a body portion is referred to as “corresponding” to a location relative to a subject (e.g., a medical patient). This may mean that the location on the image of the body portion corresponds to the location on the subject of the same anatomical feature. For instance, if the ultrasound probe is positioned against a subject's abdomen, the location identified on the image of the torso may be at the abdomen if the location is meant to represent the position of the ultrasound probe relative to the subject. Also, distances illustrated on the image of the body portion may be said to correspond to distances relative to the subject when they are the same or proportional to distances relative to the subject.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

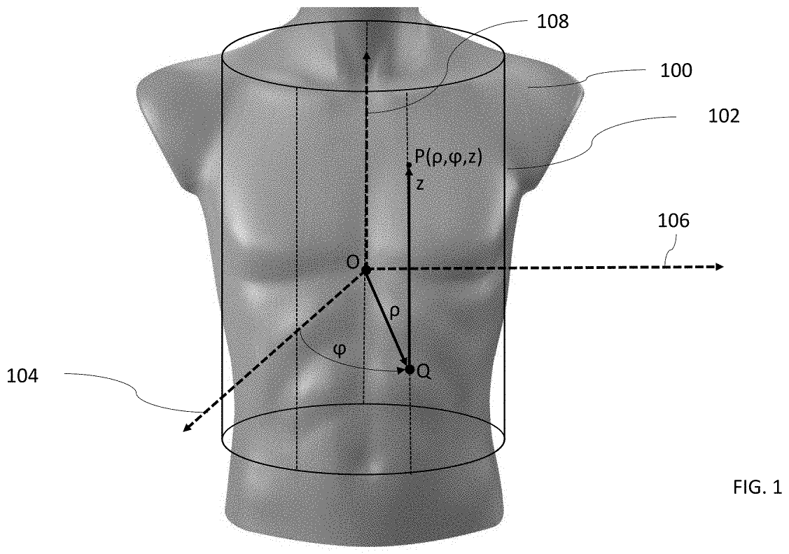

FIG. 1 illustrates an example coordinate system for a canonical body portion, which in FIG. 1 is a canonical torso, in accordance with certain embodiments described herein. A canonical torso may be a torso that is representative of physical torsos across a general population or across a portion of the general population. For example, the canonical torso may have approximately average characteristics (e.g., height, girth, etc.) across the population or a specific portion of the population. The canonical torso may be modeled by a geometric model 102. In FIG. 1, the geometric model 102 is a three-dimensional cylinder that approximates the size and shape of a 3D model of a canonical torso 100.

The geometric model 102 has a cylindrical coordinate system including a first axis 104, a second axis 106, a third axis 108, and an origin O. (For simplicity, only the positive directions of the first axis 104, the second axis 106, and the third axis 108 are shown.) The set of coordinates of a given point P on the geometric model 102 in the coordinate system includes three values (ρ,φ,z). The coordinate ρ equals the distance from the origin O to a projection of point P onto a plane formed by the first axis 104 and the second axis 106. In FIG. 1, this projection is shown as point Q. The coordinate φ equals the angle from the positive first axis 104 to the point Q. The coordinate z equals the signed distance from Q to P (i.e., the coordinate z is positive if P is above the plane formed by the first axis 104 and the second axis 106 and is negative if P is above the plane formed by the first axis 104 and the second axis 106).

To generate the geometric model 102, various three-dimensional cylinders may be projected (e.g., using CAD software) onto the 3D model of the canonical torso 100 (which may be implemented as a CAD model) such that the cylinders and the 3D model of the canonical torso 100 occupy the same three-dimensional space. Certain portions of the cylinders may be outside the 3D model of the canonical torso 100, certain portions of the cylinders may be inside the 3D model of the canonical torso 100, and/or certain portions of the cylinders may intersect with the 3D model of the canonical torso 100. The cylinder having dimensions (i.e., height and diameter), position, and orientation relative to the 3D model of the canonical torso 100 such that, compared with other cylinders, the sum of the shortest distances from each point on the 3D model of the canonical torso 100 to the cylinder is minimized, may be selected as the geometric model 102.

A given point on the 3D model of the canonical torso 100 may have a corresponding set of coordinates in the cylindrical coordinate system of the geometric model 102. In particular, the set of coordinates of the point on the geometric model 102 that is closest to a particular point on the 3D model of the canonical torso 100 may be considered the corresponding set of coordinates of the point on the 3D model of the canonical torso 100. The 3D model of the canonical torso 100 may be projected onto a two-dimensional (2D) image of the canonical torso. In particular, one or more points on the 3D model of the canonical torso 100 may be projected onto a single point on the 2D image of the canonical torso. The average of the sets of coordinates corresponding to the one or more points on the 3D model of the canonical torso 100 that are projected onto a given point on the image of the canonical torso may be considered the set of coordinates corresponding to the point on the image of the canonical torso. Various types of mappings may be used in connection with aspects of the present application. One type of mapping, referred to for simplicity as an “image-to-coordinates mapping,” may map a given point on an image of the canonical torso to a corresponding set of coordinates in the coordinate system of the geometric model 102. Another type of mapping, referred to for simplicity as a “3D image-to-coordinates mapping,” may map points on a 3D image of the 3D model of the canonical torso 100 to coordinates in the coordinate system.

A particular set of coordinates in the coordinate system of the geometric model 102 may have a corresponding point on the 3D model of the canonical torso 100. In particular, the point on the 3D model of the canonical torso 100 that is closest to a particular point on the geometric model 102 having the particular set of coordinates may be considered to be the particular set of coordinates' corresponding point on the 3D model of the canonical torso 100. Finding a point on a 2D image of a torso that corresponds to a given set of coordinates may be accomplished by first finding the point on the 3D model of the canonical torso 100 that corresponds to the given set of coordinates, as described above, and then finding the point on the 2D image of the torso to which the point on the 3D model projects when the 3D model of the canonical torso 100 is projected onto the 2D image of the torso. One type of mapping, referred to for simplicity as an “coordinates-to-image mapping,” may map a given set of coordinates in the coordinate system of the geometric model 102 to a point on an image of the torso. Another type of mapping, referred to for simplicity as a “coordinates-to-3D image mapping,” may map coordinates to points on a 3D image of the 3D model of the canonical torso 100.

It should be appreciated that while FIG. 1 shows the geometric model 102 of a canonical torso, other models of a torso may be used and models of other canonical body portions may also be used. Furthermore, there may be image-to-coordinates and coordinates-to-image mappings for the coordinate system of the model and an image of the body portion. A model of a canonical body portion may be any shape or collection of shapes that can approximate the size and shape of the canonical body portion. For example, the geometric model 102 of the canonical torso may not be a cylinder in some embodiments.

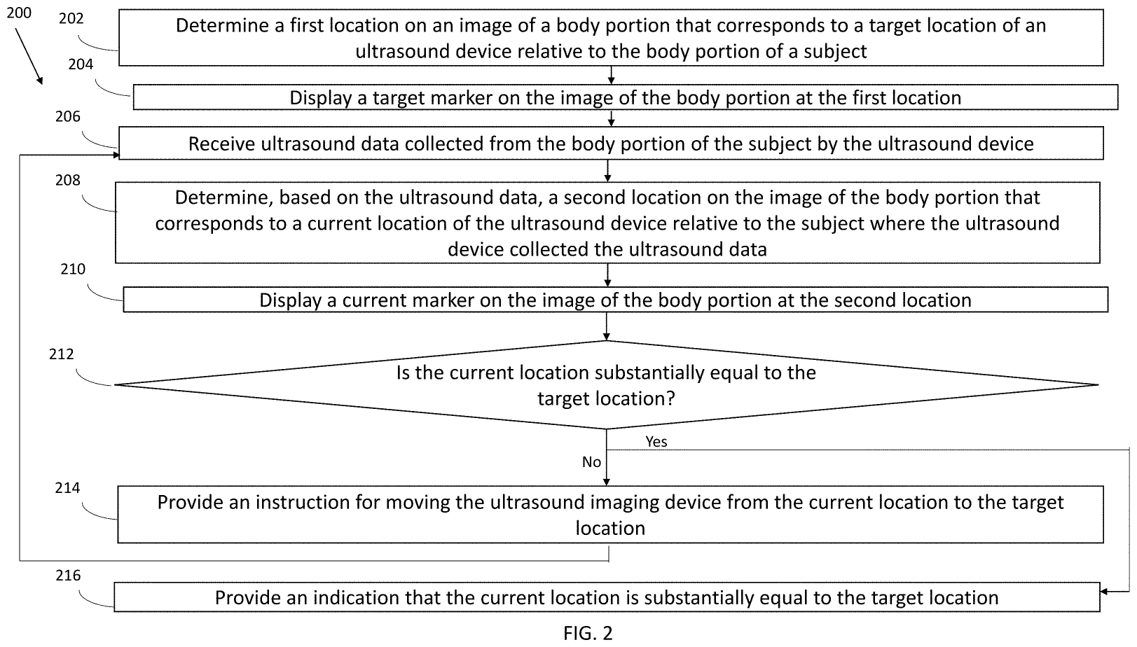

FIG. 2 illustrates an example process 200 for guiding collection of ultrasound data, in accordance with certain embodiments described herein. The process 200 may be performed by a processing device in an ultrasound system. The processing device may be, for example, a mobile phone, tablet, laptop, or server, and may be in operative communication with an ultrasound device.

In act 202, the processing device determines a first location on an image of a body portion that corresponds to a target location of an ultrasound device relative to the body portion of a subject. The target location may be a location where ultrasound data containing a target anatomical view (e.g., a parasternal long axis view of the heart) can be collected. In some embodiments, determining the first location may include determining a particular pixel or set of pixels in the image. In some embodiments, to determine the first location, the processing device may determine a target set of coordinates in a coordinate system of a model of the body portion, and then use a coordinates-to-image mapping to determine the first location on the image of the body portion that corresponds to the target set of coordinates. As an example, the target set of coordinates may be in the cylindrical coordinate system of the geometric model 102 of a torso. In some embodiments, the processing device may determine the target set of coordinates by receiving a selection of a target anatomical view from a user of the ultrasound device. For example, in some embodiments the user may select the target anatomical view from a menu of options displayed on a display screen on the processing device, or the user may type the target anatomical view into the processing device, or the user may speak the target anatomical view into a microphone on the processing device. In such embodiments, to determine the target set of coordinates, the processing device may look up the target anatomical view in a database containing associations between target anatomical views and sets of coordinates and the processing device may return the target set of coordinates associated with the target anatomical view in the database. The database may be stored on the processing device or the processing device may transmit the target anatomical view to a remote server storing the database, and the remote server may look up the target anatomical view in the database and transmit back to the processing device the target set of coordinates associated with the target anatomical view in the database. The database may be constructed by a medical professional selecting, on an image of the body portion, the location on the image that corresponds to a location on a real subject where a particular anatomical view can be collected. Once the location on the image of the body portion has been selected, the processing device may use an image-to-coordinates mapping to determine the set of coordinates in the coordinate system of the model that corresponds to that location on the image. This set of coordinates may be associated with the particular anatomical view in the database. This may be repeated for multiple anatomical views.

As another example, in some embodiments a remote medical professional may select the target anatomical view. For example, the processing device may be in wireless communication with a second processing device used by a medical professional at a different location than the user of the ultrasound device. The remote medical professional may input the target anatomical view by, for example, selecting the target anatomical view from a menu of options, by typing the target anatomical view into the second processing device, or by speaking the target anatomical view into a microphone on the second processing device, and the second processing device may wirelessly transmit the target anatomical view, or the target set of coordinates as determined from the database described above, to the processing device in operative communication with the ultrasound device.

As another example of selecting the target anatomical view, in some embodiments the processing device may automatically select the target anatomical view. The processing device may automatically select the target anatomical view as part of a workflow. For example, the workflow may include automatically instructing the user of the ultrasound device to collect the target anatomical view periodically. As another example, the workflow may include an imaging protocol that requires collecting multiple anatomical views. If the user selects such an imaging protocol (e.g., FAST, eFAST, or RUSH exams), the processing device may automatically select the target anatomical view, which may be an anatomical view collected as part of the imaging protocol. As another example, the processing device may be configured to only collect the target anatomical view, such as in a situation where the user of the ultrasound device receives the ultrasound device for the purpose of monitoring a specific medical condition that only requires collecting the target anatomical view. As another example, the processing device may select the target anatomical view by default.

As another example of determining the first location, in some embodiments the user may select, from a display screen on the processing device that shows an image of the body portion, the first location on the image of the body portion. To select the location on the image of the body portion, the user may click a mouse cursor on the location, or touch the location on a touch-enabled display screen. In some embodiments, a remote medical professional may select the first location on the image of the body portion. For example, the processing device may be in wireless communication with a second processing device used by a medical professional at a different location than the user of the ultrasound device. The display screen of the second processing device may display the image of the body portion, and the medical professional may click a mouse cursor on the location, or touch the location on a touch-enabled display screen. In some embodiments, the second processing device may transmit the first location to the processing device in operative communication with the ultrasound device. In some embodiments, the second processing device may use an image-to-coordinates mapping to determine the target set of coordinates corresponding to the first location selected on the image of the body portion and transmit the target set of coordinates to the processing device in operative communication with the ultrasound device. The process 200 proceeds from act 202 to act 204.

In act 204, the processing device displays a target marker on the image of the body portion at the first location determined in act 202. In some embodiments, the processing device may display on a display screen (e.g., the processing device's display screen) the image of the body portion, and superimpose the target marker on the image of the body portion at the first location determined in act 202. Various embodiments described herein reference a marker. In the embodiment of FIG. 2, as well as any of the other embodiments of the present application, a marker may be any suitable visual indicator of any suitable shape, size and color. For example, in any of the embodiments described herein—unless otherwise indicated—the marker may be an arrow, line (solid, dotted, dashed, or otherwise), dot, dash, square, circle, triangle, or any other suitable visual indicator. The process 200 proceeds from act 204 to act 206.

In act 206, the processing device receives ultrasound data collected from the body portion of the subject by the ultrasound device. The processing device may receive the ultrasound data in real-time, and the ultrasound data may therefore be collected from the current location of the ultrasound device on the subject being imaged. The ultrasound data may include, for example, raw acoustical data, scan lines generated from raw acoustical data, or one or more ultrasound images generated from raw acoustical data. In some embodiments, the ultrasound device may generate scan lines and/or ultrasound images from raw acoustical data and transmit the scan lines and/or ultrasound images to the processing device. In some embodiments, the ultrasound device may transmit the raw acoustical data to the processing device and the processing device may generate the scan lines and/or ultrasound images from the raw acoustical data. In some embodiments, the ultrasound device may generate scan lines from the raw acoustical data, transmit the scan lines to the processing device, and the processing device may generate ultrasound images from the scan lines. The ultrasound device may transmit the ultrasound data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device. The process proceeds from act 206 to act 208.

In act 208, the processing device determines, based on the ultrasound data received in act 206, a second location on the image of the body portion that corresponds to a current location of the ultrasound device relative to the subject where the ultrasound device collected the ultrasound data that was received in act 206. In some embodiments, determining the second location may include determining a particular pixel or set of pixels in the image. In some embodiments, to determine the second location, the processing device may determine, based on the ultrasound data received in act 206, a current set of coordinates in the coordinate system of the model of the canonical body portion, and use a coordinates-to-image mapping to determine the second location on the image that corresponds to the current set of coordinates. To determine the current set of coordinates based on the ultrasound data, the processing device may input the ultrasound data to a deep learning model trained to accept ultrasound data as an input and output a set of coordinates corresponding to the ultrasound data.

The deep learning model may be trained by providing it with training data, including sets of ultrasound data collected by ultrasound devices at multiple locations on subjects. The ultrasound data collected at each location may be labeled with a set of coordinates corresponding to the location on the subject where the ultrasound device collected the ultrasound data. For example, as discussed above, a particular location on a body portion of a subject may correspond to a particular location on an image of the body portion, and a particular location on an image of a body portion may correspond to a particular set of coordinates. As a simplified example for illustration purposes only, the torso of a subject may be divided into a two-dimensional grid of 25 locations, which the location at the upper left of the grid having coordinates (0,0), the location at the upper right of the grid having coordinates (0,5), the location at the lower left of the grid having coordinates (5,0), and the location at the lower right of the grid having coordinates (5,5). As another example, a user who is collecting training ultrasound data may place the ultrasound device at a particular location on a subject, find a location on an image of a body portion that corresponds to the location on the subject, and then determine a set of coordinates correspond to the location on the image of the body portion using an image-to-coordinates mapping. As another example, a certain anatomical structure, based on its position within a canonical body portion, may be associated with a particular set of coordinates in a coordinate system of a model of the canonical body portion. Thus, the heart may have one set of coordinates and the gallbladder may have another set of coordinates, for example. Ultrasound data collected from a particular anatomical structure may be labeled with that anatomical structure's corresponding set of coordinates. Multiple instances of ultrasound data labeled with corresponding sets of coordinates may be used to train a deep learning model, and the deep learning model may thereby learn to determine, based on inputted ultrasound data, a set of coordinates corresponding to the ultrasound data. In some embodiments, the processing device may receive a selection of the subject's body type (e.g., height, girth, male/female, etc.), and the deep learning model may use information about the subject's body type when determining the set of coordinates to output for given ultrasound data. In other words, the body type information may be used by the deep learning model to normalize outputs of the deep learning to the model of the canonical body portion. The deep learning model may be a convolutional neural network, a random forest, a support vector machine, a linear classifier, and/or any other deep learning model. The process 200 proceeds from act 208 to act 210.

In act 210, the processing device displays a current marker on the image of the body portion at the second location determined in act 208. In some embodiments, the processing device may display on a display screen (e.g., the processing device's display screen) the image of the body portion, and the processing device may superimpose the current marker on the image at the second location determined in act 208. In some embodiments, the target marker (displayed in act 204) and the current marker (displayed in act 208) may be displayed on the same image. The target marker may have a different form (e.g., color, outline, shape, symbol, size, etc.) than the current marker. In some embodiments, the image of the body portion may show anatomical structures, and displaying the current marker may include highlighting, on the image, the anatomical structure where the ultrasound device is currently located. Similarly, displaying the target marker may include highlighting, on the image, the anatomical structure that is targeted for ultrasound data collection. It should be appreciated that the current marker and the target marker may be displayed and updated as the ultrasound device is collecting ultrasound data. For example, if the ultrasound device moves to a new location relative to the subject and collects new ultrasound data, the processing device may display the current marker at a new location relative to the image of the body portion based on the new ultrasound data. This may be considered real-time updating of the location of the current marker. It should be appreciated that the processing device may not require any optical image/video of the actual ultrasound device on the subject in order to determine the location on the image of the body portion for displaying the current marker. In other words, the processing device may determine how to display the current marker on the image of the body portion based on the ultrasound data received in act 206, rather than based on any optical image/video data. Indeed, in some embodiments, the image of the body portion may not be an optical image/video of the subject being imaged, but may be, for example, a stylized/cartoonish image of the body portion or an optical image/video of a generic body portion (e.g., a model of the body portion or another individual's body portion). Furthermore, while in some embodiments the current marker may be an image of the ultrasound device, in other embodiments the current marker may not be an image of the ultrasound device. For example, the current marker may be a symbol or a shape. The process 200 proceeds from act 210 to act 212.

In act 212, the processing device determines if the current location of the ultrasound device relative to the subject is substantially equal to the target location of the ultrasound device relative to the subject. To do this, in some embodiments, the processing device may determine if the current set of coordinates determined in act 202 are substantially equal to the target set of coordinates determined in act 208. If the current set of coordinates are substantially equal to the target set of coordinates, then the ultrasound device may be at a location relative to the subject where a target anatomical view can be collected. If the current set of coordinates are not substantially equal to the target set of coordinates, then the ultrasound device may need to be moved to a location relative to the subject where the target anatomical view can be collected. Determining if the current set of coordinates is substantially equal to the target set of coordinates may include determining if each respective coordinate of the current set of coordinates is within a certain threshold value of the corresponding coordinate of the target set of coordinates. For example, in cylindrical coordinates, the processing device may determine if the ρ coordinate of the current set of coordinates is within a certain threshold value of the ρ coordinate of the target set of coordinates, if the φ coordinate of the current set of coordinates is within a certain threshold value of the φ coordinate of the target set of coordinates, and if the z coordinate of the current set of coordinates is within a certain threshold value of the z coordinate of the target set of coordinates. If the processing device determines that the current set of coordinates are substantially equal to the target set of coordinates, the process 200 proceeds to act 216. If the processing device determines that the current set of coordinates are not substantially equal to the target set of coordinates, the process 200 proceeds to act 214.

In act 214, the processing device provides an instruction for moving the ultrasound device. In some embodiments, the processing device may provide the instruction based on the current set of coordinates and the target set of coordinates. In particular, the processing device may provide an instruction determined to substantially eliminate differences between the current set of coordinates and the target set of coordinates. For example, consider current set of coordinates in the cylindrical coordinate system of the geometric model 102 having a φ coordinate that is smaller in value than the φ coordinate of the target set of coordinates. In such an example, the processing device may determine that the ultrasound device must move in the medial-lateral direction in order to substantially eliminate the difference between the y coordinates of the current set of coordinates and the target set of coordinates. As another example, consider current set of coordinates in the in the cylindrical coordinate system of FIG. 1 having a z coordinate that is smaller in value than the z coordinate of the target set of coordinates. In such an example, the processing device may determine that the ultrasound device must move in the superior-inferior direction in order to substantially eliminate the difference between the z coordinates of the current set of coordinates and the target set of coordinates. As another example, both the φ and the z coordinates of the current set of coordinates and the target set of coordinates may differ. In some embodiments, the processing device may first provide instructions to substantially eliminate differences in the z coordinates and then provide instructions to substantially eliminate differences in the y coordinates (or vice versa). In some embodiments, the processing device may provide an instruction to substantially eliminate differences in the z coordinates and in the y coordinates simultaneously. Substantially eliminating the difference between two values may include minimizing the difference between two values until the two values are within a threshold value. In some embodiments, the processing device may provide an instruction determined to substantially eliminate differences between the current set of coordinates and an intermediate target set of coordinates, where the intermediate target set of coordinates may be coordinates for a known anatomical structure between the current location and the final location. For example, if the target location is the heart and the current location is the bladder, the intermediate location may be the abdominal aorta.

To provide the instruction, the processing device may display the instruction on a display screen (e.g., a display screen of the processing device). In some embodiments, the processing device may display text corresponding to the instruction (e.g., “Move the probe in the superior direction”). In some embodiments, the processing device may display an arrow corresponding to the instruction (e.g., an arrow pointing the superior direction relative to the subject). Once the processing device has provided the instruction, the user of the ultrasound device may move the ultrasound device to a new location in response to the instructions. The process 200 proceeds from act 214 back to acts 206, 208, 210, 212, and optionally 214, in which the processing device receives new ultrasound data (e.g., from the new current location), determines whether the new current location is substantially equal to the target location, and optionally provides a new instruction for moving the ultrasound device if the new current location is still not equal to the target location.

Act 216 proceeds if the processing device determines, at act 212, that the current location of the ultrasound device is substantially equal to the target location of the ultrasound device. For example, the processing device may determine at act 212 that the current set of coordinates and the target set of coordinates are substantially equal. In act 216, the processing device provides an indication that the current location is substantially equal to the target location. Because this condition may mean that the ultrasound device is at a location relative to the subject where a target anatomical view can be collected, the indication may equivalently provide an indication that the ultrasound device is correctly positioned. To provide the indication, the processing device may display the indication on a display screen (e.g., a display screen of the processing device). In some embodiments, the processing device may display text (e.g., “The probe is positioned correctly”). In some embodiments, the processing device may display a symbol (e.g., a checkmark). In some embodiments, the processing device may play audio (e.g., audio of “The probe is positioned correctly”).

It should be appreciated that certain steps in the process 200 may be omitted and/or occur in different orders than shown in FIG. 2. For example, in some embodiments, act 204 may be omitted, such that the target marker is not shown on a display screen. Instead, the instructions provided in act 214 may be sufficient for instructing the user how to move the ultrasound device. In some embodiments, act 214 may be omitted, such that an instruction for moving the ultrasound device is not provided. Instead, the display of the target marker (in act 204) and the current marker (in act 212) on the display screen may be sufficient for indicating to the user how to move the ultrasound device. In some embodiments, the process 200 may proceed from act 214 to act 202, to determine whether a new target location has been selected. In some embodiments, acts 202 and 204 may occur after acts 206 and 208. In some embodiments, act 216 may be omitted, as it may be clear from the display of the current marker and the target marker when the current location is substantially equal to the target location. In some embodiments, only acts 206-210 may occur, such that only the current marker corresponding to the current location of the ultrasound device may be displayed. In some embodiments, only acts 202-210 may occur, such that only the current marker corresponding to the current location of the ultrasound device and the target marker corresponding to the target location of the ultrasound device may be displayed.

One non-limiting embodiment of FIG. 2 is now described. A user may select, on a processing device in communication with an ultrasound device, a cardiac imaging preset. The processing device may display a stylized image of a generic human torso and a filled-in dot on the cardiac region of the torso in the image, where the filled-in dot represents the target location for the ultrasound device. The user may place the ultrasound device on the subject's abdomen. The ultrasound device may collect ultrasound data from the subject's abdomen and transmit the ultrasound data to the processing device. The processing device may input the ultrasound data to a deep learning model, which may output that the ultrasound data was collected at the user's abdomen. Based on this determination, the processing device may display an open dot on the abdominal region of the torso in the image, where the open dot represents the current location of the ultrasound device. The processing device may also determine that the user needs to move the ultrasound device in the superior direction relative to the subject in order to move the ultrasound device to the target location, and may display an arrow in the superior direction relative to the subject. In response to the arrow, the user may move the ultrasound device from the subject's abdomen to the subject's cardiac region. The ultrasound device may collect ultrasound data from the subject's cardiac region and transmit the ultrasound data to the processing device. The processing device may input the ultrasound data to the deep learning model, which may output that the ultrasound data was collected at the user's cardiac region. Based on this determination, the processing device may display a checkmark, indicating that the ultrasound device is at the target location.

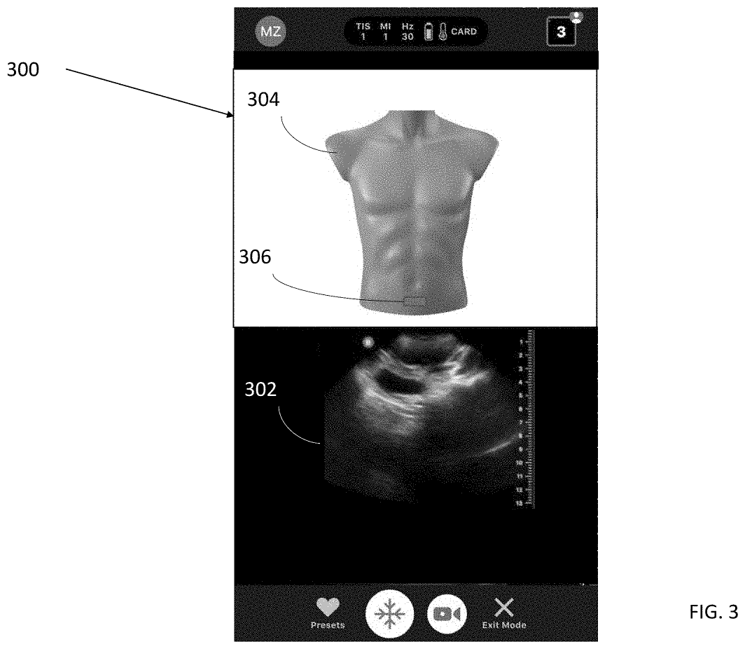

FIGS. 3-7, 9-12, 15, and 17-19 illustrate example graphical user interface (GUI)s 300-700, 900-1200, 1500, and 1700-1900, respectively, that may be displayed on a display screen of a processing device in an ultrasound system, in accordance with certain embodiments described herein. The processing device may be, for example, a mobile phone, tablet, laptop, or server, and may be in operative communication with the ultrasound device, such as over a wired communication link (e.g., over Ethernet, a Universal Serial Bus (USB) cable or a Lightning cable) and/or over a wireless communication link (e.g., over a BLUETOOTH, WiFi, or ZIGBEE wireless communication link).

The GUI 300 of FIG. 3 may be displayed on the processing device in real-time during collection of ultrasound data by the ultrasound device. The GUI 300 includes an ultrasound image 302, an image of a torso 304, and a current marker 306 indicating the current location of an ultrasound device relative to a subject where it collected ultrasound data.

In some embodiments, the ultrasound image 302 may be generated from ultrasound data collected by the ultrasound device. Further description of collection of ultrasound data may be found with reference to act 206, and further description of determining the location of the current marker 306 and displaying the current marker 306 may be found with reference to acts 208 and 210. The image of the torso 304 may be an image of the specific subject being imaged or a generic image of the torso (e.g., an image of a model torso or an image of another subject's torso). The image of the torso 304 may be, for example, an optical image, an exterior image, an image generated by electromagnetic radiation, a photographic image, a non-photographic image, and/or non-ultrasound image. In FIGS. 3-7, the image of the torso 304 is a non-photographic image of a model torso.

In some embodiments, as new ultrasound data is received at the processing device, the processing device may determine a new current set of coordinates corresponding to the new ultrasound data and show the current marker 306 at a new location on the image of the torso 304, as well as a new ultrasound image generated from the new ultrasound data, in real-time. Thus, as the ultrasound device moves, the current marker 306 may move on the image of the torso 304 as well. It should be appreciated that the appearance of the current marker 306 in FIG. 3 is not limiting and may have other shapes, colors, outlines, symbols, sizes, etc. It will further be appreciated the relative positions of the various features shown on the display screen (e.g., the ultrasound image 302 and the torso image 304) are illustrative in nature and that other arrangements are also contemplated. For example, the ultrasound image 302 may be displayed above the torso image 304.

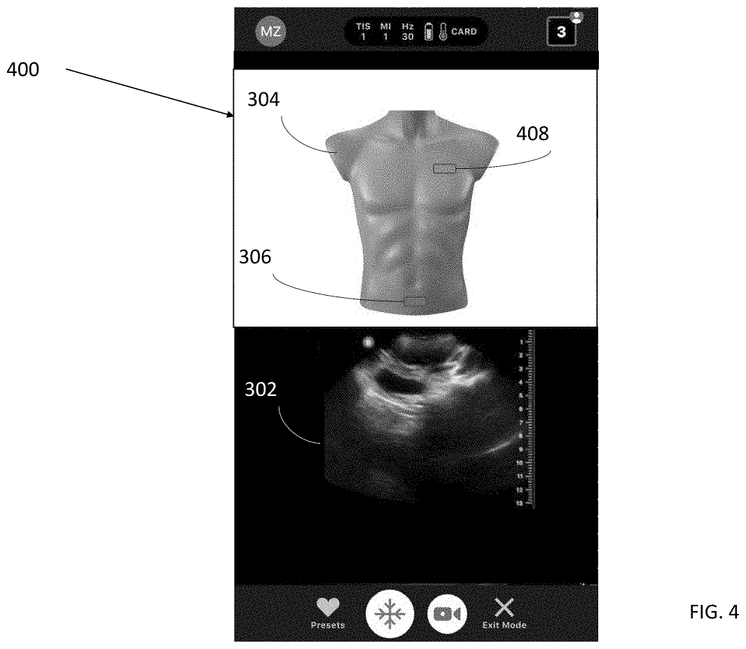

The GUI 400 of FIG. 4 differs from the GUI 300 in that the GUI 400 includes a target marker 408 indicating a target location of an ultrasound device relative to a subject. Further description of determining the location of the target marker 408 and displaying the target marker 408 may be found with reference to acts 202 and 204.

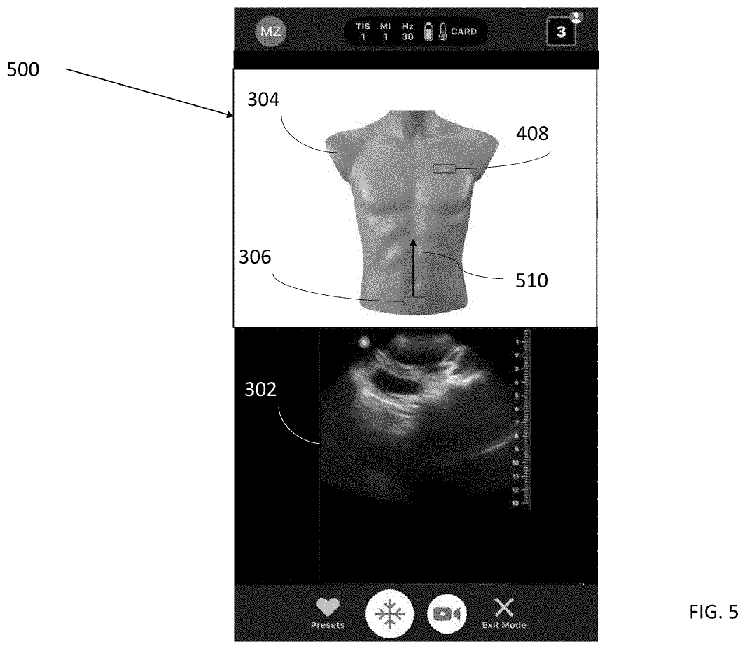

The GUI 500 of FIG. 5 differs from the GUI 400 in that the GUI 500 includes an instruction 510 for moving the ultrasound device. In the example of FIG. 5, the instruction 510 is an arrow extending in a substantially superior direction relative to the image of the torso 304 from the current marker 306. The instruction 510 may be provided by the processing device to instruct a user of the ultrasound device to move the ultrasound device in the direction shown by the instruction 510 (i.e., in the superior direction relative to the subject). It should be appreciated that moving the ultrasound device from its current location in a substantially superior direction relative to the subject may move the ultrasound device closer to a location where the ultrasound device may be able to collect a target anatomical view.

The instruction 510 may be provided by the processing device to substantially eliminate differences between the current set of coordinates corresponding to the ultrasound image 302 currently being collected by the ultrasound device and an intermediate target set of coordinates corresponding to an intermediate location between the current location and final target location of the ultrasound device. For example, if the current location of the ultrasound device is the bladder and the target location is the heart, the intermediate location may be abdominal aorta. In the example of FIG. 5, the current set of coordinates may have a z coordinate that is smaller in value than the z coordinate of the intermediate target set of coordinates. In such an example, the processing device may determine that the ultrasound device must move in the superior direction relative to the subject in order to substantially eliminate the difference between the z coordinates of the current set of coordinates and the intermediate target set of coordinates. Once the difference between the z coordinates of the current set of coordinates and the intermediate target set of coordinates has been substantially eliminated, the processing device may cease to provide the instruction 510. Further description of providing the instruction 510 may be found with reference to act 214.

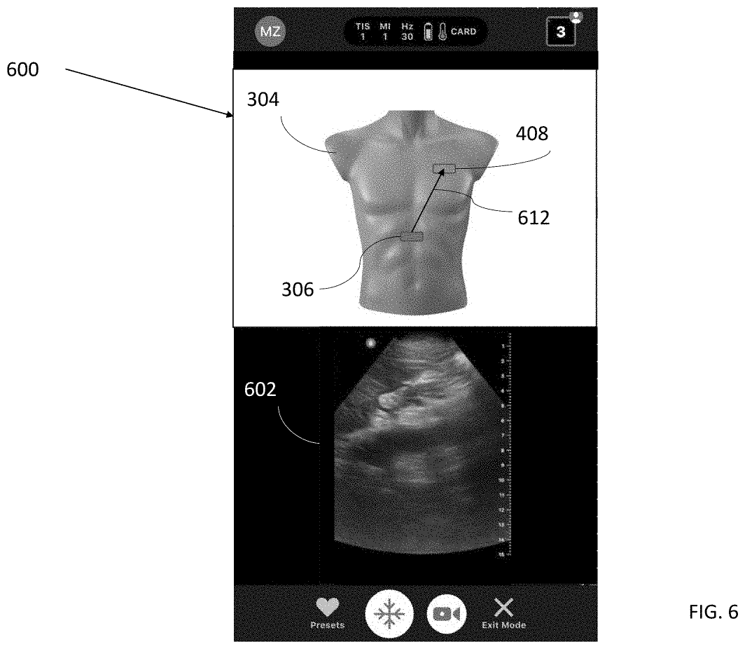

The GUI 600 of FIG. 6 differs from the GUI 500 in that the GUI 600 includes another instruction 612 for moving the ultrasound device. The instruction 612 is an arrow extending in a substantially lateral and superior directions relative to the image of the torso 304 from the current marker 306. Additionally, as can be seen in FIG. 6, the current marker 306 has moved in the superior direction relative to the image of the torso 304 from the location shown by the current marker 306 in FIG. 5. The user may have moved the ultrasound device in response to the instruction 510 until moving the ultrasound device in the direction shown by the instruction 510 did not substantially eliminate differences between the current set of coordinates and the intermediate target set of coordinates. The processing device may then provide the instruction 612 to instruct the user to further move the ultrasound device in the superior and lateral directions. In other words, moving the ultrasound device from the current location on the subject to the location where the ultrasound device may collect the target anatomical view may require moving the ultrasound device in both the superior and lateral directions. The GUI 600 also includes an ultrasound image 602 that may have been collected at the current location of the ultrasound device.

The instruction 612 may be provided by the processing device to substantially eliminate differences between the current set of coordinates corresponding to the ultrasound image 602 and the target set of coordinates corresponding to a target anatomical view. In the example of FIG. 6, the current set of coordinates may have a φ coordinate that is smaller in value than the φ coordinate of the target set of coordinates and a z coordinate that is smaller in value than the z coordinate of the target set of coordinates. In such an example, the processing device may determine that the ultrasound device must move in the lateral and superior directions relative to the subject in order to substantially eliminate the differences between the z and φ coordinates of the current set of coordinates and the target set of coordinates. Once the differences between the z and φ coordinates of the current set of coordinates and the target set of coordinates have been substantially eliminated, the processing device may cease to provide the instruction 612. Further description of providing instructions may be found with reference to acts 212 and 214. It should be appreciated that while FIGS. 5 and 6 show the instructions 510 and 612 in the form of arrows, other forms for the instructions 510 and 612 are possible, such as text. Other instructions, such as moving the ultrasound device in other directions relative to the subject, may also be provided, and instructions may be provided in different orders (e.g., first instructing the user to move the ultrasound device in the lateral direction and then in the superior direction). In some embodiments, a user may use the current marker 306 and the target marker 408 to determine how to move the ultrasound device to the location on the subject where the ultrasound device may collect the target anatomical view. In particular, the user may view movement of the current marker 306 in response to movement of the ultrasound device and continue to move the ultrasound device until the current marker 306 is at the target marker 408. In such embodiments, instructions such as instruction 510 and 612 may not be displayed.

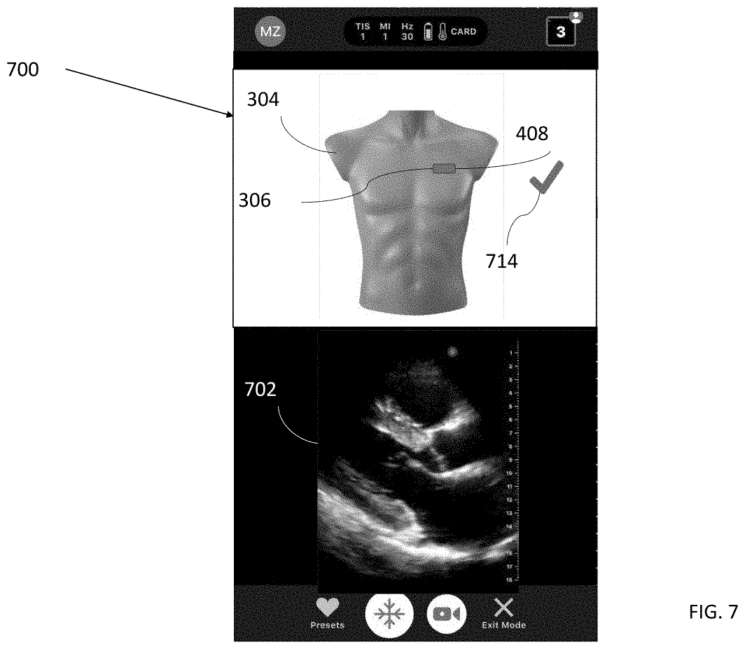

The GUI 700 of FIG. 7 differs from the GUI 600 in that the GUI 700 includes an indicator 714 that the current location of the ultrasound device relative to the subject is substantially equal to the target location of the ultrasound device relative to the subject. Additionally, the GUI 700 includes an ultrasound image 702 that may have been collected at the current location of the ultrasound device. In some embodiments, this may be determined when the set of coordinates determined from the ultrasound image 702 currently being collected by the ultrasound device and the target set of coordinates corresponding to a target anatomical view are substantially equal. This condition may mean that the ultrasound device is at a location on the subject where a target anatomical view may be collected. The processing device may provide the indicator 714 if each respective coordinate of the current set of coordinates is within a certain threshold value of the corresponding coordinate of the target set of coordinates. It should be noted that in FIG. 7, the current marker 306 and the target marker 408 are at substantially the same location on the image of the torso 304, and no further instructions are provided for moving the ultrasound device. It should also be noted that while the indicator 714 is in the form of a checkmark, other indicators 714 are possible, such as text or other symbols. Further description of providing an indicator that the current set of coordinates are substantially equal to the target set of coordinates may be found with reference to act 216.

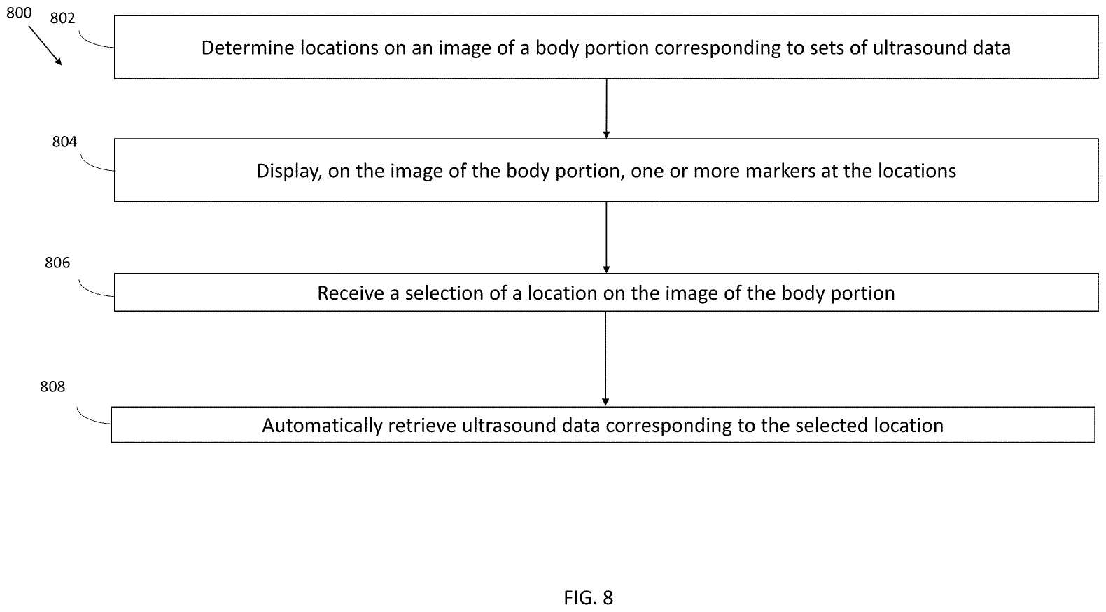

FIG. 8 illustrates an example process 800 for retrieving ultrasound data, in accordance with certain embodiments described herein. The process 800 may be performed by a processing device in an ultrasound system. Using the process 800, a user may be able to view ultrasound data based on selecting a location on an image of a body portion

In act 802, the processing device determines locations on an image of a body portion corresponding to sets of ultrasound data. Each location on the image of the body portion may correspond to a location relative to the body portion of the subject where a set of ultrasound data was collected. In some embodiments, determining the locations may include determining particular pixels or sets of pixels in the image. In some embodiments, to determine the locations, the processing device may determine sets of coordinates in a coordinate system of a model of the body portion (e.g., the geometric model 102 of the canonical torso), where each set of coordinates corresponds to a set of ultrasound data. The ultrasound device may have collected the ultrasound data during one or more imaging sessions, and the processing device may receive a selection of sets of ultrasound data collected during these imaging sessions. For example, the sets of ultrasound data may include multiple ultrasound images collected during an imaging session, such as ultrasound images from different portions of the abdominal aorta (i.e., proximal, mid, and distal abdominal aorta) collected during an abdominal aortic aneurysm scan, or ultrasound images containing different anatomical views collected during an imaging protocol (e.g., FAST, eFAST, or RUSH protocols). The sets of ultrasound data may have been collected in the past, and the ultrasound data may be saved in memory. The ultrasound data may include, for example, raw acoustical data, scan lines generated from raw acoustical data, or one or more ultrasound images generated from raw acoustical data. To determine the sets of coordinates corresponding to the sets of ultrasound data, the processing device may input each set of ultrasound data to a deep learning model trained to accept ultrasound data as an input and output a set of coordinates corresponding to the ultrasound data. In some embodiments, the processing device may input the sets of ultrasound data to the deep learning model upon selection of the sets of ultrasound data. In some embodiments, the processing device (or another processing device) may have previously inputted the sets of ultrasound data to the deep model and saved the sets of coordinates to a database which the processing may access in act 802 to determine the set of coordinates. Further description of determining a set of coordinates from ultrasound data may be found with reference to act 208. The process 800 proceeds from act 802 to act 804.

In act 804, the processing device displays one or more markers at the locations on the image of the body portion that were determined in act 802. In some embodiments, the processing device may display on a display screen (e.g., the processing device's display screen) an image of the body portion (e.g., a torso) and the processing device may use a coordinates-to-image mapping to determine the locations on the image that correspond to the sets of coordinates, and superimpose markers at those locations on the image. In some embodiments, the markers may be discrete markers. In some embodiments, the marker may be a path. For example, the locations determined in act 802, when displayed on the image of the body portion, may appear as a substantially continuous path. This may occur, for example, if an ultrasound device collected ultrasound data substantially continuously while traveling along a path relative to a subject (e.g., a path along the abdominal aorta). As another example, the processing device may generate a path by interpolating paths between the locations on the image corresponding to the sets of coordinates determined in act 802. In some embodiments, the processing device may display both a path indicating movement of the ultrasound device along the path and discrete markers superimposed on the path. The process 800 proceeds from act 804 to act 806.

In act 806, the processing device receives a selection of a location on the image of the body portion. A user may make the selection, for example, by clicking a mouse or touching a touch-enabled display screen. In some embodiments, the selection of the location may be a selection of a discrete marker displayed at a location on an image of the body portion. In such embodiments, the processing device may determine the set of coordinates corresponding to that location using an image-to-coordinates mapping. In some embodiments, the selection of the location may be a selection of a location along a path that was displayed in act 804, and the processing device may determine a set of coordinates corresponding to the selected location using an image-to-coordinates mapping. In some embodiments, it may be possible for a user to select a location on the image of the body portion that does not correspond to ultrasound data in the sets of ultrasound data from act 802. In particular, the selected location may correspond to a set of coordinates (based on an image-to-coordinates mapping), and that set of coordinates may not have been determined in act 802 as corresponding to any of the sets of ultrasound data. As an example, the path may be generated by interpolating paths between the locations on the image corresponding to the sets of coordinates determined in act 802, such that there may not be ultrasound data in the sets of ultrasound data that correspond to locations on the interpolated paths. If a user selects a location that does not correspond to ultrasound data in the sets of ultrasound data, in some embodiments the processing device may select a location that is closest to the selected location and which corresponds to a set of coordinates that does correspond to collected ultrasound data. In some embodiments, if a user selects a location that does not correspond to ultrasound data in the sets of ultrasound data, the processing device may return an error and not display ultrasound data in act 808. With regards to determining whether the selected location corresponds to ultrasound data in the sets of ultrasound data, as described above with reference to act 802, the processing device determines sets of coordinates corresponding to sets of ultrasound data. The set of coordinates associated with each set of ultrasound data may be stored in a database, and the processing device may access this database to determine if a selected set of coordinates corresponds to a set of ultrasound data.

In some embodiments, one or more of the sets of coordinates determined in act 802 may correspond to anatomical views. For example, the processing device may access a database containing associations between target anatomical views and sets of coordinates. The processing device may receive a selection from a user (who may be the same user who collected the ultrasound data, or a medical professional who may be remote from the use who collected the ultrasound data) of one of these anatomical views. For example, in some embodiments the user may select the target anatomical view from a menu of options displayed on a display screen on the processing device, or the user may type the target anatomical view into the processing device, or the user may speak the target anatomical view into a microphone. In such embodiments, the processing device may look up the anatomical view in the database and select the set of coordinates associated with the selected anatomical view in the database.

In some embodiments, the processing device may highlight the selection. In embodiments in which the processing device displayed a marker in act 804 that corresponds to the selected set of coordinates, the processing device may highlight the marker corresponding to the selected set of coordinates (e.g., by changing a color, size, shape, symbol, etc.). In embodiments in which a marker corresponding to the selected set of coordinates was not displayed in act 804 (e.g., a path was displayed but no specific markers), the processing device may display a marker at a location on an image of the body portion corresponding to the selected set of coordinates (e.g., using a coordinates-to-image mapping). In some embodiments, the processing device may display text corresponding to an anatomical view corresponding to the selected set of coordinates (e.g., “parasternal long axis view of the heart”). For example, the processing device may access a database containing associations between target anatomical views and sets of coordinates to determine the anatomical view corresponding to the selected set of coordinates. The process 800 proceeds from act 806 to act 808.

In act 808, the processing device automatically retrieves ultrasound data corresponding to the selected location of act 806. As described above with reference to act 806, a set of coordinates corresponding to the selection may be determined. As described above with reference to act 802, the processing device may determine sets of coordinates corresponding to sets of ultrasound data. The set of coordinates associated with each set of ultrasound data may be stored in a database, and the processing device may access this database to determine the ultrasound data corresponding to the selected set of coordinates and display this ultrasound data on a display screen (e.g., a display screen on the processing device). In some embodiments, the processing device may display the retrieved the ultrasound data. For example, if the set of ultrasound data is an ultrasound image, the processing device may display the ultrasound image. As another example, if the set of ultrasound data is a sequence of ultrasound images, the processing device may display the sequence of ultrasound images as a video.

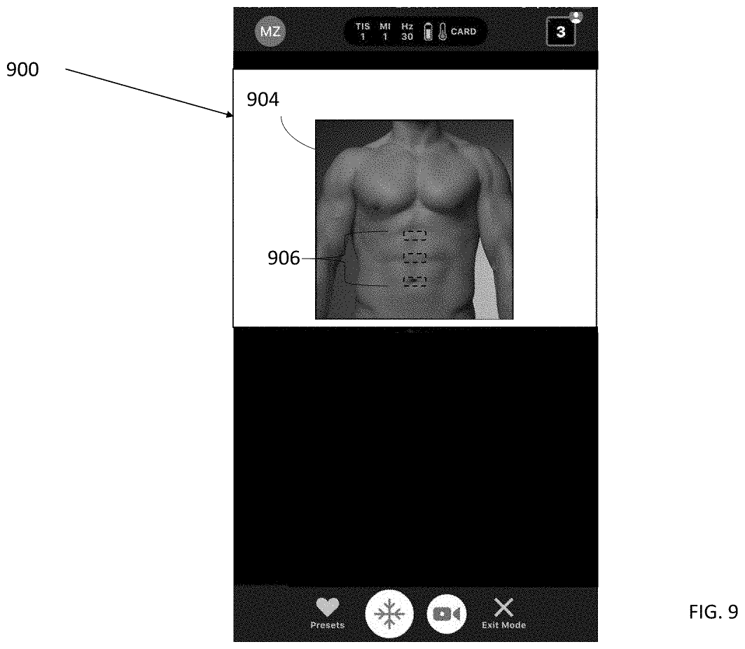

The GUI 900 of FIG. 9 includes an image of a torso 904 and markers 906 corresponding to ultrasound data that was previously collected. The image of the torso 904 may be an image of the specific subject being imaged or a generic image of the torso (e.g., an image of a model torso or an image of another subject's torso). The image of the torso 904 may be, for example, an optical image, an exterior image, an image generated by electromagnetic radiation, a photographic image, a non-photographic image, and/or non-ultrasound image. In FIGS. 9-12, 15, and 17-19, the image of the torso 904 is a photographic image of a model torso.

In some embodiments, the processing device may determine locations on the image of the torso 904 corresponding to sets of ultrasound data. Each of the markers 906 may correspond to one of these locations. Further description of determining locations for the markers 906 and displaying the markers 906 may be found with reference to acts 802 and 804. The three markers 906 shown may, for example, correspond to ultrasound images containing anatomical views of the proximal, mid, and distal abdominal aorta.

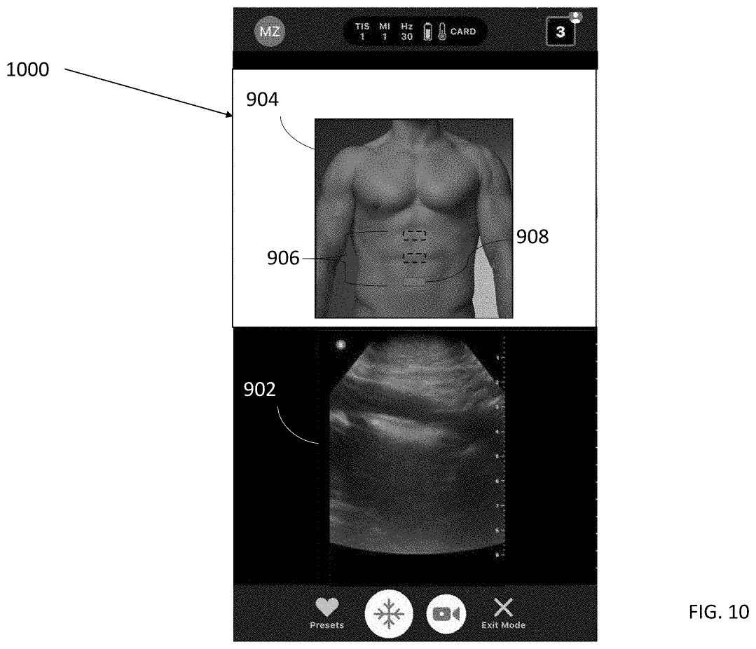

The processing device may display the GUI 1000 of FIG. 10 after selection of the marker 908 on the GUI 900. A user may select one of the markers 906 (e.g., by clicking a mouse or touching a touch-enabled display screen). The GUI 1000 highlights a selected marker 908 and displays an ultrasound image 902 corresponding to the selected marker 908. The selected marker 908 may be highlighted in any manner (e.g., by using a different color, shape, outline, symbol, size, etc., from the other markers 906). Upon selection of the marker 908, the processing device may determine that the ultrasound image 902 corresponds to the selected marker 908 and display the ultrasound image 902. Further description of selecting the marker 908 and displaying the ultrasound image 902 may be found with reference to acts 806 and 808. While an ultrasound image 902 is shown in FIG. 10, upon selection of the marker 908, the processing device may display any type of ultrasound data, such as displaying a sequence of ultrasound images as a video. In the example of FIG. 10, the markers 906 may correspond to ultrasound data collected at the proximal, mid, and distal abdominal aorta. While three markers 906 are shown in the GUI 1000, any suitable number of markers may be shown.

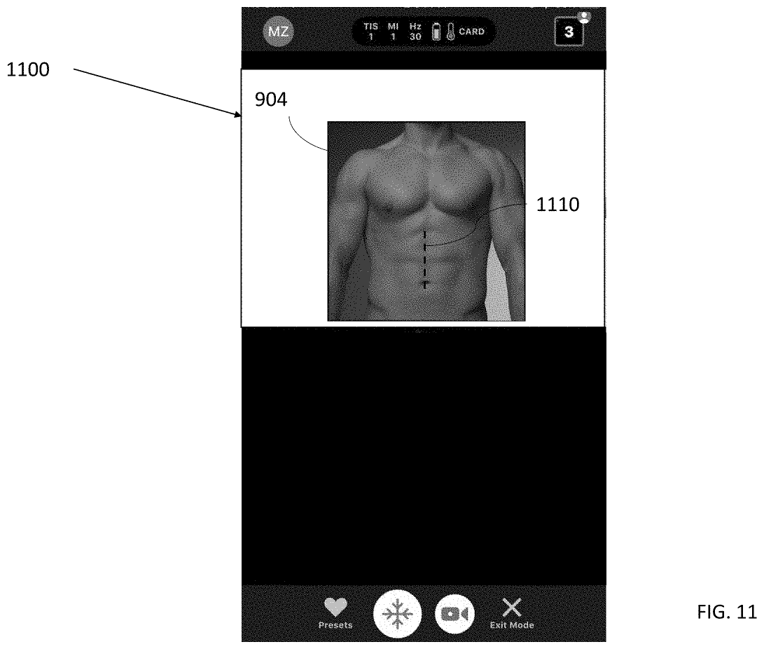

The GUI 1100 of FIG. 11 differs from the GUI 900 in that the GUI 1100 shows a path 1110 instead of the multiple markers 906 superimposed on the image of the torso 904. Further description of determining locations along the path 1110 and displaying the path 1110 may be found with reference to act 804.

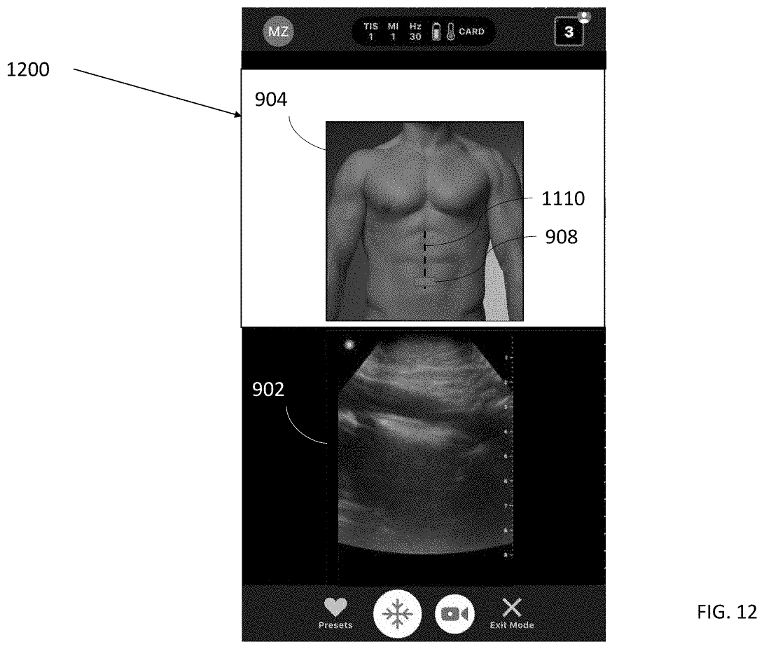

The processing device may display the GUI 1200 of FIG. 12 after selection of a location along the path 1100. The GUI 1200 displays a marker 908 at a selected location along the path 1110 and an ultrasound image 902 corresponding to the location. Further description of selecting a location on the path 1110, displaying the marker 908, and displaying the ultrasound image 902 may be found with reference to acts 806 and 808.

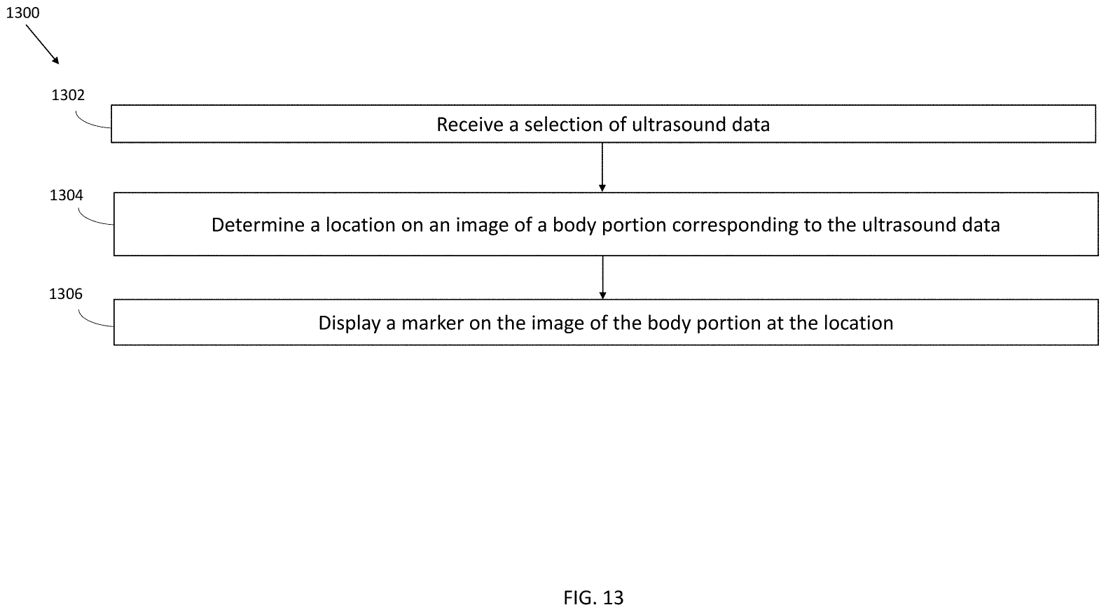

FIG. 13 illustrates an example process 1300 for retrieving ultrasound data, in accordance with certain embodiments described herein. The process 1300 may be performed by a processing device in an ultrasound system. Using the process 1300, a user may be able to view a location on an image of a body portion corresponding to ultrasound data that the user selected.

In act 1302, the processing device receives a selection of ultrasound data. The ultrasound data may include, for example, raw acoustical data, scan lines generated from raw acoustical data, or one or more ultrasound images generated from raw acoustical data. In some embodiments, the ultrasound data may be saved in memory, where the memory may be in the processing device and/or on another device. In embodiments in which the ultrasound data is saved in memory at another device, the processing device may receive the selection of ultrasound data by a user selecting a hyperlink to the ultrasound data stored at the other device, where selecting the hyperlink causes the processing device to download the ultrasound data from the other device and/or causes the processing device to access a webpage containing the ultrasound data. In some embodiments, the processing device may display thumbnails of ultrasound data, and a user may select particular ultrasound data by selecting (e.g., by clicking a mouse or touching on a touch-enabled display) a thumbnail corresponding to the ultrasound data. In some embodiments, the processing device may display a carousel through which a user may scroll to view multiple sets of ultrasound data, one after another. In some embodiments, upon selection of ultrasound data, the ultrasound data may be displayed at full size. The process 1300 proceeds from act 1302 to act 1304.

In act 1304, the processing device determines a location on an image of a body portion that corresponds to the ultrasound data selected in act 1302. The location on the image of the body portion may correspond to a location relative to the body portion of the subject where the ultrasound data selected in act 1302 was collected. In some embodiments, determining the location may include determining a particular pixel or set of pixels in the image. In some embodiments, the processing device may determine a set of coordinates in a coordinate system of a model of the body portion (e.g., the geometric model 102 of the canonical torso), where the set of coordinates corresponds to the ultrasound data selected in act 1302. To determine the set of coordinates corresponding to the ultrasound data, the processing device may input the ultrasound data to a deep learning model trained to accept ultrasound data as an input and output a set of coordinates corresponding to the ultrasound data. In some embodiments, the processing device may input the ultrasound data to the deep learning model upon selection of the ultrasound data. In some embodiments, the processing device (or another processing device) may have previously inputted the sets of ultrasound data to the deep model and saved the sets of coordinates to a database which the processing may access at act 1304 to determine the set of coordinates. In some embodiments, to determine the location on the image of the body portion that corresponds to the set of coordinates, the processing device may use a coordinates-to-image mapping to determine the location on the image that corresponds to the set of coordinates determined in act 1304. Further description of determining a set of coordinates from ultrasound data may be found with reference to act 208. The process 1300 proceeds from act 1304 to act 1306.

In act 1306, the processing device displays a marker at the location on the image of the body portion that was determined in act 1304. In some embodiments, the processing device may display on a display screen (e.g., the processing device's display screen) the image of the body portion (e.g., a torso) and the processing device may superimpose a marker on the image at the location determined in act 1304. Further description of displaying a marker may be found with reference to act 210.

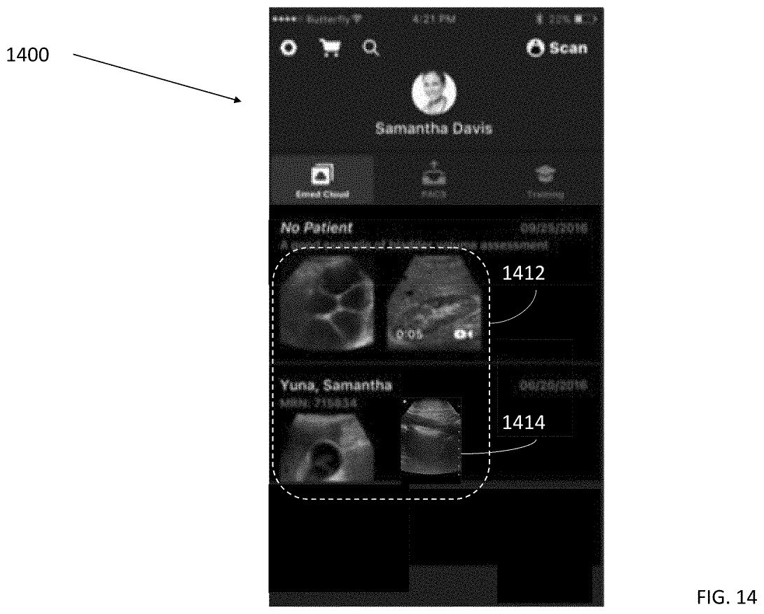

The GUI 1400 of FIG. 14 includes multiple thumbnails 1412 of ultrasound data. Each of the thumbnails 1412 shows a small-size version of an ultrasound image previously collected by the ultrasound device, or a small-size image from a sequence of ultrasound images previously collected by the ultrasound device. A user may select one of the thumbnails 1412, such as thumbnail 1414 (e.g., by clicking a mouse or touching a touch-enabled display). Further description of selecting ultrasound data may be found with reference to act 1302.

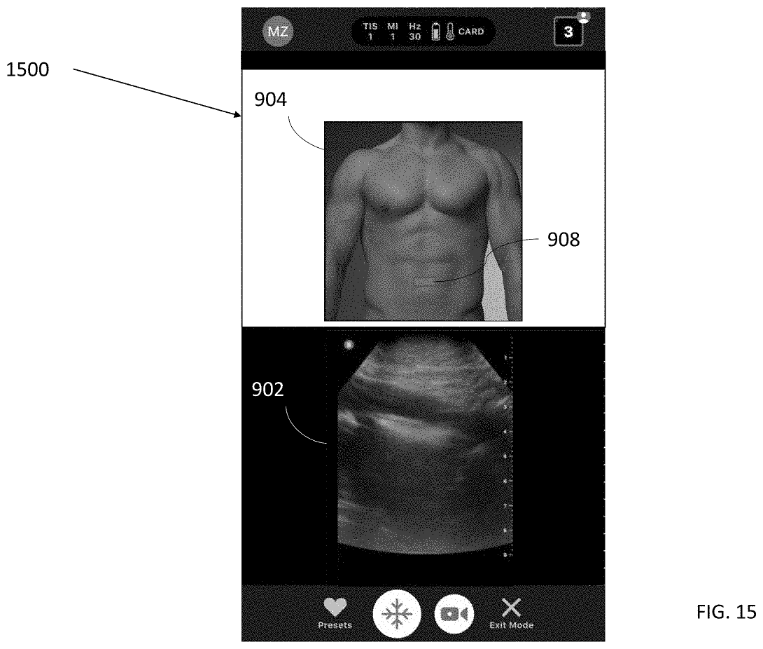

The GUI 1500 of FIG. 15 may be displayed after selection of the thumbnail 1414 from the GUI 1400. The GUI 1500 includes the ultrasound image 902, the image of the torso 904, and the marker 908. The ultrasound image 902 is a larger-size version of the ultrasound image shown by the thumbnail 1414. Further description of determining a location for the marker 908 and displaying the marker 908 may be found with reference to act 1304 and act 1306.

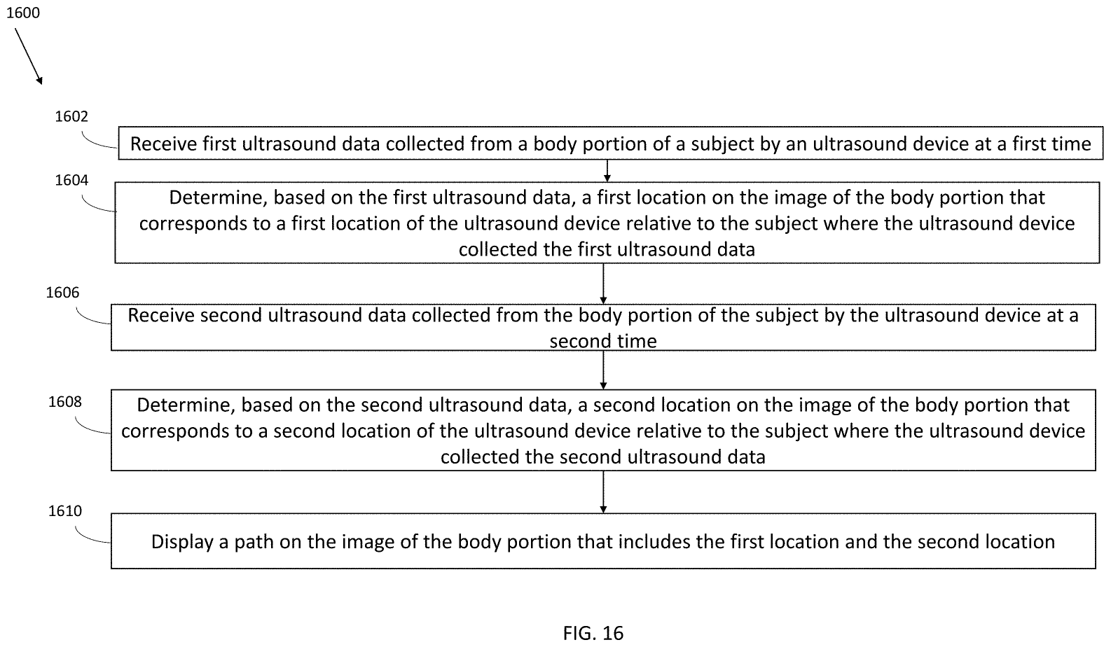

FIG. 16 illustrates an example process 1600 for collection of ultrasound data, in accordance with certain embodiments described herein. The process 1600 may be performed by a processing device in an ultrasound system. The processing device may be, for example, a mobile phone, tablet, laptop, or server, and may be in operative communication with an ultrasound device.

In act 1602, the processing device receives first ultrasound data collected from a body portion of a subject by an ultrasound device at a first time. Further description of receiving ultrasound data may be found with reference to act 206. The process 1600 proceeds from act 1602 to act 1604.

In act 1604, the processing device determines, based on the first ultrasound data received in act 1602, a first location on the image of the body portion that corresponds to a first location of the ultrasound device relative to the subject where the ultrasound device collected the first ultrasound data. Further description of determining a location on an image of a body portion that corresponds to a location of an ultrasound device relative to a subject may be found with reference to act 208. The process 1600 proceeds from act 1604 to act 1606.

In act 1606, the processing device receives second ultrasound data collected from the body portion of the subject by the ultrasound device at a second time. The second time may be after the first time. The first and second times may occur during a current imaging session. The first time may be a previous time and the second time may be the current time. Further description of receiving ultrasound data may be found with reference to act 206. The process 1600 proceeds from act 1606 to act 1608.

In act 1608, the processing device determines, based on the second ultrasound data, a second location on the image of the body portion that corresponds to a second location of the ultrasound device relative to the subject where the ultrasound device collected the second ultrasound data. Further description of determining a location on an image of a body portion that corresponds to a location of an ultrasound device relative to a subject may be found with reference to act 208. The process 1600 proceeds from act 1608 to act 1610.

In act 1610, the processing device displays a path on the image of the body portion that includes the first location and the second location determined in acts 1604 and 1606. In some embodiments, the path may include a line or another shape that proceeds through the first and second locations on the image of the body portion. In some embodiments, the path may include locations that are interpolated between the first and second locations. In some embodiments, the path may include a first marker at the first location and a second marker at the second location. In some embodiments, the path may include both a line or another shape that proceeds through the first and second locations on the image of the body portion and a first marker at the first location and a second marker at the second location. In some embodiments, the path may include one or more directional indicators (e.g., arrows) that indicate the order in which ultrasound data along the path was collected. For example, if the first time was before the second time, the path may include an arrow pointing from the first location to the second location. Further description of displaying a path and/or markers on the image of the body portion may be found with reference to acts 210 and 804.

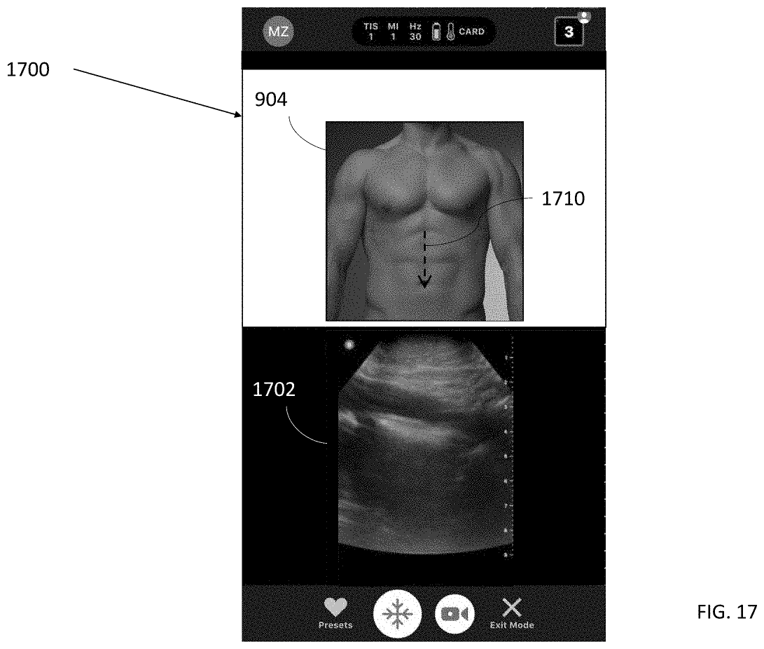

The GUI 1700 of FIG. 17 includes the image of the torso 904, a path 1710, and an ultrasound image 1702. In some embodiments, the processing device may determine locations on the image of the torso 904 corresponding to sets of ultrasound data that are collected at different times during a current imaging session. The path 1710 may include these locations. The path 1710 includes an arrow. The arrow may indicate the order in which ultrasound data was collected. For example, the arrow in FIG. 17 points from the upper abdomen to the lower abdomen, indicating that ultrasound data may be have been collected first from the upper abdomen and then from the lower abdomen. In some embodiments, one end of the path may be at a location where the first set of ultrasound data was collected, and the other end of the path may be at a location where the current (e.g., most recent) set of ultrasound data was collected. The arrow on the path 1710 may point from the end of the path at the location where the first set of ultrasound data was collected to the end of the path at the location where the current set of ultrasound data was collected. The ultrasound image 1702 may be the current set of ultrasound data or one image from the current set of ultrasound data. The processing device may update the path 1710 as further ultrasound data is collected from further locations. Further description of determining locations for the path 1710 and displaying the path 1710 may be found with reference to acts 1604-1610.

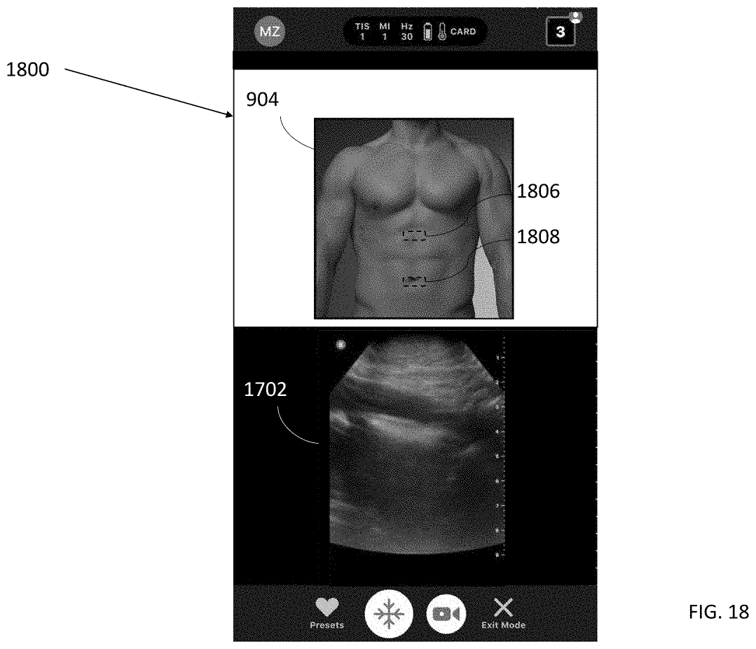

The GUI 1800 of FIG. 18 includes the image of the torso 904 and markers 1806 and 1808. In some embodiments, the processing device may determine locations on the image of the torso 904 corresponding to sets of ultrasound data that are collected at different times during a current imaging session. Each of the markers 1806 and 1808 may correspond to one of these locations. One of the markers 1806 and 1808 may correspond to the location where the current (e.g., most recent) set of ultrasound data was collected. The ultrasound image 1702 may be the current set of ultrasound data or one image from the current set of ultrasound data. Further description of determining locations for the markers 1806 and 1808 and displaying the markers 1806 and 1808 may be found with reference to acts 1604-1610.

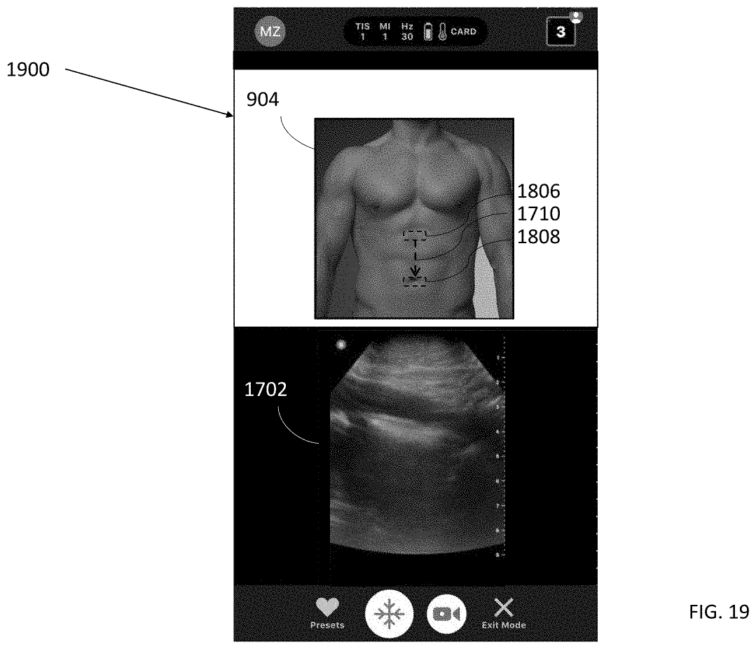

The GUI 1900 of FIG. 19 includes the image of the torso 904, the path 1710, and the markers 1806 and 1808.

While in FIGS. 18-19, only two markers 1806 and 1808 are displayed, it should be appreciated that more or fewer markers may be displayed, depending on how many sets of ultrasound data have been collected.

While FIGS. 3-7, 9-12, 15, and 17-19 illustrate a torso, and such embodiments may include a processing device using a coordinate system of a model of a canonical torso, the graphical user interfaces depicted in FIGS. 3-7, 9-12, 15, and 17-19 may also be used for other body portions, such as the abdomen, arm, breast, chest, foot, genitalia, hand, head, leg, neck, pelvis, thorax, torso, or entire body. While the graphical user interfaces of FIGS. 3-7, 9-12, 15, and 17-19 display an ultrasound image, in some embodiments the graphical user interfaces may display a sequence of ultrasound images as a video. In some embodiments, rather than displaying a two-dimensional image of a body portion (e.g., the images of the torso 304 or 904), the processing device may display a three-dimensional (3D) image of the body portion, and the processing device may superimpose markers (e.g., the marker 908) on the three-dimensional image using a coordinates-to-3D image mapping. The 3D image of the body portion may be viewed, for example, using 3D glasses.

In any of the embodiments described herein where coordinates are determined using a model of a body portion, one of multiple models of the same body portion may be used. For example, there may be different models for different heights, girths, male/female, etc. Prior to a processing device determining coordinates, the body type of the subject may be inputted into the processing device so that the appropriate model of the body portion can be used.

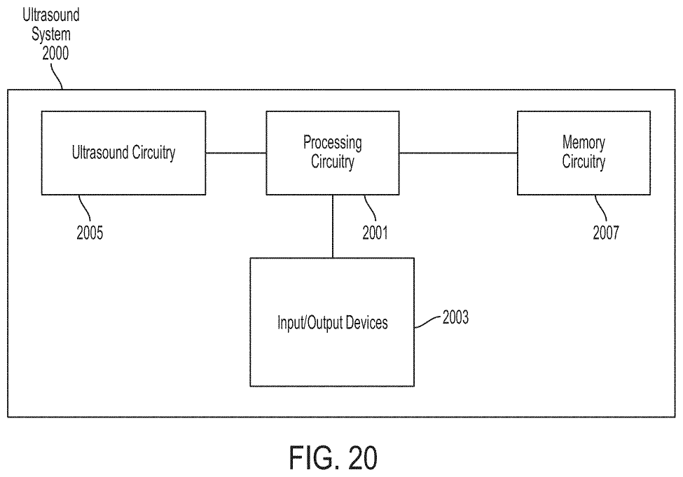

FIG. 20 illustrates a schematic block diagram illustrating aspects of an example ultrasound system 2000 upon which various aspects of the technology described herein may be practiced. For example, one or more components of the ultrasound system 2000 may perform any of the processes (e.g., the processes 200, 800, 1300, or 1600) described herein. As shown, the ultrasound system 2000 includes processing circuitry 2001, input/output devices 2003, ultrasound circuitry 2005, and memory circuitry 2007.

The ultrasound circuitry 2005 may be configured to generate ultrasound data that may be employed to generate an ultrasound image. The ultrasound circuitry 2005 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 2005 (e.g., transmit circuitry, receive circuitry, control circuitry, power management circuitry, and processing circuitry) to form a monolithic ultrasound imaging device.

The processing circuitry 2001 may be configured to perform any of the functionality described herein. The processing circuitry 2001 may include one or more processors (e.g., computer hardware processors). To perform one or more functions, the processing circuitry 2001 may execute one or more processor-executable instructions stored in the memory circuitry 2007. The memory circuitry 2007 may be used for storing programs and data during operation of the ultrasound system 2000. The memory circuitry 2007 may include one or more storage devices such as non-transitory computer-readable storage media. The processing circuitry 2001 may control writing data to and reading data from the memory circuitry 2007 in any suitable manner.

In some embodiments, the processing circuitry 2001 may include specially-programmed and/or special-purpose hardware such as an application-specific integrated circuit (ASIC). For example, the processing circuitry 2001 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

The input/output (I/O) devices 2003 may be configured to facilitate communication with other systems and/or an operator. Example I/O devices 2003 that may facilitate communication with an operator include: a keyboard, a mouse, a trackball, a microphone, a touch screen, a printing device, a display screen, a speaker, and a vibration device. Example I/O devices 2003 that may facilitate communication with other systems include wired and/or wireless communication circuitry such as BLUETOOTH, ZIGBEE, Ethernet, WiFi, and/or USB communication circuitry.

It should be appreciated that the ultrasound system 2000 may be implemented using any number of devices. For example, the components of the ultrasound system 2000 may be integrated into a single device. In another example, the ultrasound circuitry 2005 may be integrated into an ultrasound imaging device that is communicatively coupled with a processing device that includes the processing circuitry 2001, the input/output devices 2003, and the memory circuitry 2007.

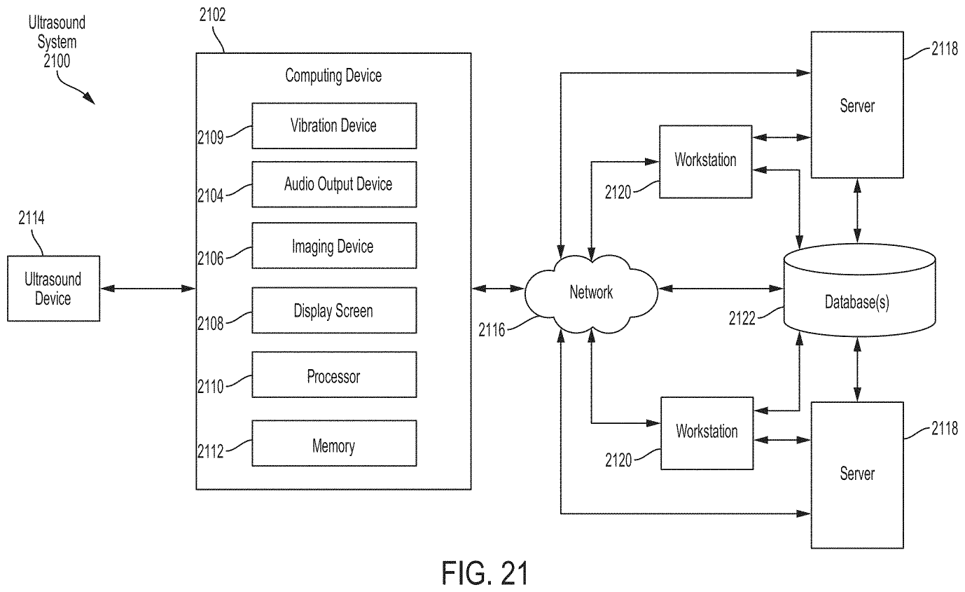

FIG. 21 illustrates a schematic block diagram illustrating aspects of another example ultrasound system 2100 upon which various aspects of the technology described herein may be practiced. For example, one or more components of the ultrasound system 2100 may perform any of the processes (e.g., the processes 200, 800, 1300, or 1600) described herein. As shown, the ultrasound system 2100 includes an ultrasound imaging device 2114 in wired and/or wireless communication with a processing device 2102 (which may correspond to any of the processing devices described above). The processing device 2102 includes an audio output device 2104, an imaging device 2106, a display screen 2108, a processor 2110, a memory 2112 (which may correspond to any of the memories described above), and a vibration device 2109. The processing device 2102 may communicate with one or more external devices over a network 2116. For example, the processing device 2102 may communicate with one or more workstations 2120, servers 2118, and/or databases 2122.

The ultrasound imaging device 2114 may be configured to generate ultrasound data that may be employed to generate an ultrasound image. The ultrasound imaging device 2114 may be constructed in any of a variety of ways. In some embodiments, the ultrasound imaging device 2114 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data.

The processing device 2102 may be configured to process the ultrasound data from the ultrasound imaging device 2114 to generate ultrasound images for display on the display screen 2108. The processing may be performed by, for example, the processor 2110. The processor 2110 may also be adapted to control the acquisition of ultrasound data with the ultrasound imaging device 2114. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

Additionally (or alternatively), the processing device 2102 may be configured to perform any of the processes (e.g., the processes 200, 800, 1300, or 1600) described herein (e.g., using the processor 2110). As shown, the processing device 2102 may include one or more elements that may be used during the performance of such processes. For example, the processing device 2102 may include one or more processors 2110 (e.g., computer hardware processors) and one or more articles of manufacture that include non-transitory computer-readable storage media such as the memory 2112. The processor 2110 may control writing data to and reading data from the memory 2112 in any suitable manner. To perform any of the functionality described herein, the processor 2110 may execute one or more processor-executable instructions stored in one or more non-transitory computer-readable storage media (e.g., the memory 2112), which may serve as non-transitory computer-readable storage media storing processor-executable instructions for execution by the processor 2110.

In some embodiments, the processing device 2102 may include one or more input and/or output devices such as the audio output device 2104, the imaging device 2106, the display screen 2108, and the vibration device 2109. The audio output device 2104 may be a device that is configured to emit audible sound such as a speaker. The imaging device 2106 may be configured to detect light (e.g., visible light) to form an image such as a camera. The display screen 2108 may be configured to display images and/or videos such as a liquid crystal display (LCD), a plasma display, and/or an organic light emitting diode (OLED) display. The vibration device 2109 may be configured to vibrate one or more components of the processing device 2102 to provide tactile feedback. These input and/or output devices may be communicatively coupled to the processor 2110 and/or under the control of the processor 2110. The processor 2110 may control these devices in accordance with a process being executed by the processor 2110 (such as the processes 200, 800, 1300, or 1600). Similarly, the processor 2110 may control the audio output device 2104 to issue audible instructions and/or control the vibration device 2109 to change an intensity of tactile feedback (e.g., vibration) to issue tactile instructions. Additionally (or alternatively), the processor 2110 may control the imaging device 2106 to capture non-acoustic images of the ultrasound imaging device 2114 being used on a subject to provide an operator of the ultrasound imaging device 2114 an augmented reality interface.

It should be appreciated that the processing device 2102 may be implemented in any of a variety of ways. For example, the processing device 2102 may be implemented as a handheld device such as a mobile smartphone or a tablet. Thereby, an operator of the ultrasound imaging device 2114 may be able to operate the ultrasound imaging device 2114 with one hand and hold the processing device 2102 with another hand. In other examples, the processing device 2102 may be implemented as a portable device that is not a handheld device such as a laptop. In yet other examples, the processing device 2102 may be implemented as a stationary device such as a desktop computer.

In some embodiments, the processing device 2102 may communicate with one or more external devices via the network 2116. The processing device 2102 may be connected to the network 2116 over a wired connection (e.g., via an Ethernet cable) and/or a wireless connection (e.g., over a WiFi network). As shown in FIG. 21, these external devices may include servers 2118, workstations 2120, and/or databases 2122. The processing device 2102 may communicate with these devices to, for example, off-load computationally intensive tasks. For example, the processing device 2102 may send an ultrasound image over the network 2116 to the server 2118 for analysis (e.g., to identify an anatomical feature in the ultrasound) and receive the results of the analysis from the server 2118. Additionally (or alternatively), the processing device 2102 may communicate with these devices to access information that is not available locally and/or update a central information repository. For example, the processing device 2102 may access the medical records of a subject being imaged with the ultrasound imaging device 2114 from a file stored in the database 2122. In this example, the processing device 2102 may also provide one or more captured ultrasound images of the subject to the database 2122 to add to the medical record of the subject. For further description of ultrasound imaging devices and systems, see U.S. patent application Ser. No. 15/415,434 titled “UNIVERSAL ULTRASOUND IMAGING DEVICE AND RELATED APPARATUS AND METHODS,” filed on Jan. 25, 2017 (and assigned to the assignee of the instant application), which is incorporated by reference herein in its entirety. Aspects of the technology described herein relate to the application of automated image processing techniques to analyze images, such as ultrasound images. In some embodiments, the automated image processing techniques may include machine learning techniques such as deep learning techniques. Machine learning techniques may include techniques that seek to identify patterns in a set of data points and use the identified patterns to make predictions for new data points. These machine learning techniques may involve training (and/or building) a model using a training data set to make such predictions.

Deep learning techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images or sets of raw acoustical data that are each labeled with a set of coordinates in a coordinate system of a canonical body portion. In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images.

Once the training data has been created, the training data may be loaded to a database (e.g., an image database) and used to train a neural network using deep learning techniques. Once the neural network has been trained, the trained neural network may be deployed to one or more processing devices. It should be appreciated that the neural network may be trained with any number of sample patient images, although it will be appreciated that the more sample images used, the more robust the trained model data may be.

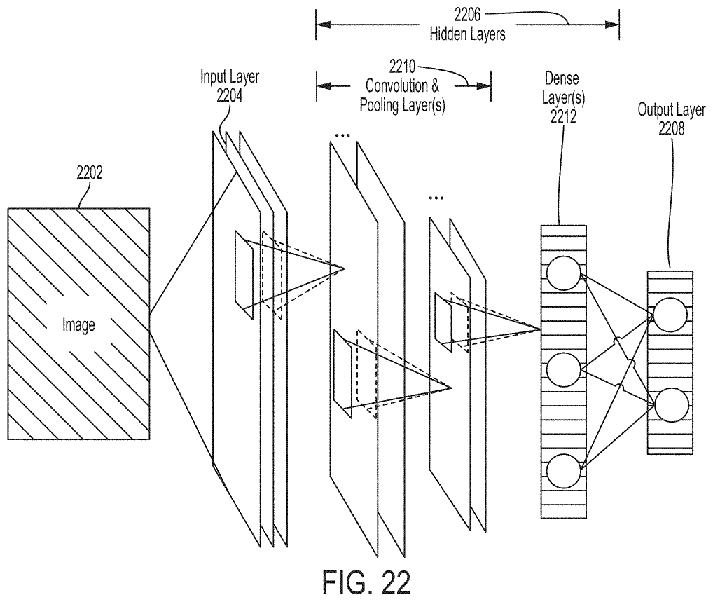

In some applications, a neural network may be implemented using one or more convolution layers to form a convolutional neural network. An example convolutional neural network is shown in FIG. 22 that is configured to analyze an image 2202. As shown, the convolutional neural network includes an input layer 2204 to receive the image 2202, an output layer 2208 to provide the output, and a plurality of hidden layers 2206 connected between the input layer 2204 and the output layer 2208. The plurality of hidden layers 2206 includes convolution and pooling layers 2210 and dense layers 2212.

The input layer 2204 may receive the input to the convolutional neural network. As shown in FIG. 22, the input the convolutional neural network may be the image 2202. The image 2202 may be, for example, an ultrasound image.

The input layer 2204 may be followed by one or more convolution and pooling layers 2210. A convolutional layer may include a set of filters that are spatially smaller (e.g., have a smaller width and/or height) than the input to the convolutional layer (e.g., the image 2202). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 2210 may be followed by dense layers 2212. The dense layers 2212 may include one or more layers each with one or more neurons that receives an input from a previous layer (e.g., a convolutional or pooling layer) and provides an output to a subsequent layer (e.g., the output layer 2208). The dense layers 2212 may be described as “dense” because each of the neurons in a given layer may receive an input from each neuron in a previous layer and provide an output to each neuron in a subsequent layer. The dense layers 2212 may be followed by an output layer 2208 that provides the output of the convolutional neural network. The output may be, for example, a set of coordinates corresponding to the image 2202.

It should be appreciated that the convolutional neural network shown in FIG. 22 is only one example implementation and that other implementations may be employed. For example, one or more layers may be added to or removed from the convolutional neural network shown in FIG. 22. Additional example layers that may be added to the convolutional neural network include: a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

For further description of deep learning techniques, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application), which is incorporated by reference herein in its entirety. In any of the embodiments described herein, instead of/in addition to using a convolutional neural network, a fully connected neural network may be used.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising a processing device in operative communication with an ultrasound device, the processing device configured to:
determine, based on first ultrasound data collected from a body portion of a subject by the ultrasound device, a first location on an image of a body portion, wherein the first location on the image of the body portion corresponds to a current location of the ultrasound device relative to the body portion of the subject where the ultrasound device collected the first ultrasound data; and
display a first marker on the image of the body portion at the first location.

2. The apparatus of claim 1, wherein the processing device is configured, when displaying the first marker on the image of the body portion, to display the first marker on a display screen of the processing device.

3. The apparatus of claim 1, wherein the processing device is further configured to receive the first ultrasound data from the ultrasound device.

4. The apparatus of claim 3, wherein the processing device is further configured to update the first location of the first marker as further ultrasound data is received at the processing device from the ultrasound device.

5. The apparatus of claim 1, wherein the processing device is further configured to:
determine a second location on the image of the body portion, wherein the second location relative to the image of the body portion corresponds to a target location of the ultrasound device relative to the body portion of the subject; and
display a second marker on the image of the body portion at the second location.

6. The apparatus of claim 5, wherein the processing device is configured, when determining the second location, to receive a selection of the second location on the image of the body portion.

7. The apparatus of claim 5, wherein the processing device is configured, when displaying the second marker, to display the second location on a display screen of the processing device.

8. The apparatus of claim 5, wherein the processing device is further configured to receive a selection of an anatomical view associated with the target location.

9. The apparatus of claim 5, wherein the processing device is further configured to provide an instruction for moving the ultrasound device from the current location to the target location.

10. The apparatus of claim 5, wherein the processing device is further configured to provide an indication that the current location is substantially equal to the target location.

11. The apparatus of claim 1, wherein the processing device is further configured to:
determine, based on second ultrasound data collected from the body portion of the subject by the ultrasound device at a past time, a second location on the image of the body portion, wherein the second location on the image of the body portion corresponds to a past location of the ultrasound device relative to the body portion of the subject where the ultrasound device collected the second ultrasound data; and
display a path on the image of the body portion that includes the first location and the second location.

12. The apparatus of claim 1, wherein the body portion comprises a torso.

13. An apparatus, comprising processing circuitry configured to:
receive a selection of a location on an image of a body portion; and
automatically retrieve ultrasound data that was collected by an ultrasound device at a location relative to a subject corresponding to the selected location.

14. The apparatus of claim 13, wherein the processing circuitry is further configured to:
display, on the image of the body portion, one or more markers at a plurality of locations on the image of the body portion.

15. The apparatus of claim 14, wherein the processing circuitry is further configured to:
determine the plurality of locations on the image of the body portion, wherein each respective location of the plurality of locations corresponds to a location relative to the body portion of a subject where an ultrasound device collected a respective set of ultrasound data of a plurality of sets of ultrasound data.

16. The apparatus of claim 15, wherein the processing circuitry is further configured to receive a selection of the plurality of sets of ultrasound data.

17. The apparatus of claim 15, wherein the plurality of sets of ultrasound data comprise:
a set of ultrasound data containing an anatomical view of a proximal abdominal aorta;
a set of ultrasound data containing an anatomical view of a mid abdominal aorta; and
a set of ultrasound data containing an anatomical view of a distal abdominal aorta.

18. The apparatus of claim 14, wherein the processing circuitry is configured, when displaying the one or more markers at the plurality of locations, to display a plurality of discrete markers at each of the plurality of locations.

19. The apparatus of claim 18, wherein the processing circuitry is configured, when receiving the selection of the location on the image of the body portion, to receive a selection of a marker of the plurality of discrete markers.

20. The apparatus of claim 19, wherein the processing circuitry is configured, when retrieving the ultrasound data corresponding to the selected location, to retrieve ultrasound data that was collected at a location relative to the subject corresponding to a location of the selected marker on the image of the body portion.

21. The apparatus of claim 14, wherein the processing circuitry is configured, when displaying the one or more markers at the plurality of locations, to display a path along the plurality of locations.

22. The apparatus of claim 21, wherein the processing circuitry is configured, when receiving the selection of the location on the image of the body portion, to receive a selection of a location along the path.

23. The apparatus of claim 22, wherein the processing circuitry is configured, when retrieving the ultrasound data corresponding to the selected location, to retrieve ultrasound data that was collected at a location relative to the subject corresponding to the selected location along the path.

24. The apparatus of claim 21, wherein the path extends along an abdominal aorta of the body portion in the image.

25. The apparatus of claim 13, wherein the body portion comprises a torso.

26. An apparatus, comprising processing circuitry configured to:
receive a selection of ultrasound data;
determine a location on an image of a body portion corresponding to a location relative to the body portion of a subject where an ultrasound device collected the ultrasound data; and
display, on the image of the body portion, a marker at the determined location.
